(19) United States
(12) Patent Application Publication
(10) Pub. No.: US 2022/0165363 A1
(43) Pub. Date: May 26, 2022

(54) DE NOVO COMPARTMENT DECONVOLUTION AND WEIGHT ESTIMATION OF TUMOR TISSUE SAMPLES USING DECODER

(71) Applicant: The University of North Carolina at Chapel Hill, Chapel Hill, NC (US)

(72) Inventors: Jen Jen Yeh, Chapel Hill, NC (US); Xianlu (Laura) Peng, Chapel Hill, NC (US)

(73) Assignee: The University of North Carolina at Chapel Hill, Chapel Hill, NC (US)

(21) Appl. No.: 17/441,138

(22) PCT Filed: Mar. 23, 2020
(86) PCT No.: PCT/US2020/024343

§ 371 Date: Sep. 20, 2021

Related U.S. Application Data
(60) Provisional application No. 62/821,824, filed on Mar. 21, 2019.

(51) Int. Cl. G16B 40/30; G16B 20/20; G06F 17/16; G16H 50/20

(52) U.S. Cl. CPC G16B 40/30; G16H 50/20; G06F 17/16; G16B 20/20

(57) ABSTRACT

Provided are methods for de novo deconvoluting of datasets with multiple samples and/or estimating compartment weights for single samples. In some embodiments, the methods include applying the process or processes to a dataset with multiple samples or to a single sample, whereby the dataset is deconvoluted and/or a compartment weight is estimated. In some embodiments, the dataset relates to RNA sequence and/or gene expression data, optionally tumor RNA sequence and/or gene expression data, wherein the tumor is in some embodiments a pancreatic tumor, a prostate cancer, a bladder cancer, or a breast cancer.

### CROSS REFERENCE TO RELATED APPLICATION

The presently disclosed subject matter claims the benefit of U.S. Provisional Patent Application Ser. No. 62/821,824, filed Mar. 21, 2019, the disclosure of which incorporated herein by reference in its entirety.

### GOVERNMENT INTEREST

This invention was made with government support under grant numbers CA199064 and CA211000 awarded by the National Institutes of Health. The government has certain rights in the invention.

### BACKGROUND

Tumor samples are mixtures of distinct cell populations that contribute to intra-tumor heterogeneity, including immune, stroma, and normal cells (Marusyk et al., 2012; Yadav & De, 2015). Therefore, with bulk tumor samples, the analysis of tumor gene expression can be significantly confounded by the presence of nonneoplastic cell types, while the contribution of the tumor microenvironment is difficult to separate. Although laser-capture microdissection (LCM) and single-cell sequencing techniques strive to tackle these problems, both of them present certain limitations. LCM is labor intensive and may influence the quality of the microdissected tissue for further analysis (Espina et al., 2007; Chung & Shen, 2015). Single-cell sequencing is still expensive, computing resource heavy, and currently limited by the lack of comprehensive cell-sorting biomarkers (Wu et al., 2014; Hague et al., 2017).

To eliminate the need of relying on LCM or single-cell-based techniques, a plethora of computational strategies have been developed to deconvolve the mixed signal present in a bulk tumor sample using RNA gene expression, DNA copy number data, or DNA methylation data. Algorithms based on DNA copy-number alterations, e.g., ABSOLUTE (Carter et al., 2012), and DNA methylation profiles, e.g., MethylPurify (Zheng et al., 2014) and InfiniumPurify (Zheng et al., 2017), focus on inferring tumor purity, while expression-based deconvolution methods mainly handle estimation of compartment fractions, as well as extraction of compartment-specific expression profiles (Yadav & De, 2015). However, the current expression-based deconvolution methods still pose a number of limitations. Some methods are limited to the presupposition of a certain combination of compartments, such as DeMix (tumor and normal; Ahn et al., 2013), UNDO (tumor and stroma; Wang et al., 2015), and ESTIMATE (tumor, stroma, and immune; Yoshihara et al., 2013). Other methods, such as DeconRNAseq (Gong & Szustakowski, 2013) and CIBERSORT (Newman et al., 2015), provide the flexibility to measure any number of specific compartments. However, they require knowledge of the pure expression of compartments as the reference, which is difficult to obtain in practice. Similarly, DSA (Zhong et al., 2013) requires lists of marker genes. However, frequently the exact compartments in a sample are unknown, and samples are inherently heterogenous. Therefore, the incomplete knowledge of the number of compartments may hamper the accuracy of calculations of the compartment proportions. In addition, like many other quadratic programming-based algorithms, DSA (Zhong et al., 2013) has a minimum sample size requirement to perform the estimation, imposing a need for larger data sets.

Pancreatic ductal adenocarcinoma (PDAC) is characterized by relatively low tumor purity and large amounts of desmoplastic stroma. Therefore, identifying tumor-specific alterations in PDAC is a continuing challenge. To perform virtual microdissection and study compartment-specific signatures, deconvolved bulk PDAC samples were previously deconvolved by adapting the nonnegative matrix factorization (NMF) algorithm (Moffitt et al., 2015). As a result, two tumor-specific (Basal-like and Classical) and two stroma-specific (Activated and Normal) subtypes were identified, together with exocrine, endocrine, and immune factors. Like other standard NMF applications, the number of factors (K) that the input matrix may be decomposed into must be determined as a priority. Although the performance of NMF at different K may be evaluated by silhouette and cophenetic correlation coefficient, this evaluation assumes the exclusive classification of each sample into one of the K clusters (Brunet et al., 2004; Bailey et al., 2016), which may not be as biologically clear cut. Previously, K was empirically determined by dedicated manual association of biological relevance to each factor at multiple trial runs of K, which can be time consuming and resource intensive (Moffitt et al., 2015). Thus, developing a streamlined framework that is able to automatically determine K is very appealing and will have potential applicability to the bulk tumor sample deconvolution of any cancer type.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides a method for de novo deconvoluting a dataset with multiple samples and/or estimating compartment weight for a single sample. In some embodiments, the method comprising applying the following steps to a dataset with multiple samples or to a single sample: using a nonnegative matrix factorization (NMF) algorithm to calculate a NMF seed training configured to calculate a stable gene weight seed (W″), wherein W″ is a 50*×\tilde{K} matrix of 50*\tilde{K} rows of genes and \tilde{K} columns of factor, completed for R repetitions resulting in R data partitions; executing an NMF algorithm for each of R data partitions; calculating a gene weight matrix for a current number (K) of factors; and executing a final NMF algorithm to generate a gene weight matrix W′ (a 5000*\tilde{K} matrix of 5000 rows of genes and \tilde{K} columns of factors), and compartment weight matrix H′ (a K×M matrix of \tilde{K} rows of factors and M columns of samples), whereby the dataset is deconvoluted and/or a compartment weight is estimated.

In some embodiments, the R repetitions is about 10,000 or greater. In some embodiments, the NMF algorithm for each of the R data partitions comprises an unsupervised NMF executed with at least 20 randomly initialized instances of NMF using a multiplicative update NMF solver for ten steps using a built-in NMF function in MATLAB (R2017b).

In some embodiments, the method further comprises calculating a consensus matrix, wherein the consensus matrix represents a frequency of the genes to be determined as the top genes, and wherein the consensus matrix is used for hierarchical clustering to yield \tilde{K} gene clusters. In some embodiments, the calculation of the gene weight matrix comprises ranking genes in a matrix in descending order of a weight difference between a current factor weight and a largest weight in the rest of the factors, wherein a top 50 genes for any factor are recorded in a gene-by-gene consensus matrix C (50*\tilde{K}×50*\tilde{K}).

In some embodiments, the method comprises using a non-negative least square (NNLS) algorithm to find:

arg minₕ_{ᵢ}∥W′\tilde{h}ᵢ−a′ᵢ∥₂subject to \tilde{h}ᵢ≥0,

where \tilde{h}ᵢ is the ith column/sample in \tilde{H} (a \tilde{K}×M matrix) to be determined, a′ᵢ is the ith column/sample in A′ (a 5000×M matrix, wherein \tilde{H} is considered to record the final compartment weights for each factor at the current number of factors (\tilde{K}) from the de novo deconvolution.

In some embodiments, the method further comprises using a NNLS algorithm to find:

arg min_{w}_{ᵢ}∥\tilde{H}\tilde{w}ⱼ−aⱼ∥₂subject to \tilde{W}ⱼ≥0,

where \tilde{w}ⱼ is the jth row/gene in \tilde{W} (a N×\tilde{K} matrix) to be determined, and as is the jth row/gene in A (a N×M matrix), wherein \tilde{W} is considered to record the final gene weights for each factor at the current number of factors (\tilde{K}) from the de novo deconvolution, wherein gene weight matrix (\tilde{W}) is further used for ranking of genes, calculation of factor scores, and/or annotation of compartments.

In some embodiments, the dataset comprises RNA sequence and/or gene expression data. In some embodiments, the RNA sequence and/or gene expression data is from a tumor, optionally ATACseq data. In some embodiments, the tumor is any tumor, such as a pancreatic tumor, a prostate cancer, a bladder cancer, or a breast cancer. In some embodiments, the tumor is from a subject, optionally a human subject. In some embodiments, the dataset is derived from any one of Tables 1-6, or any combination thereof. In some embodiments, the dataset is derived from Data Table 1.

In some embodiments, the presently disclosed subject matter provides a method of diagnosing a cancer and/or identifying a tumor type in a subject, optionally, where the tumor cannot be identified through pathology. In some embodiments, the method comprises: providing a sample from a subject to be diagnosed; and performing a method for de novo deconvoluting a dataset with multiple samples and/or estimating compartment weight for a single sample in accordance with the presently disclosed subject matter on the sample from the subject, wherein a cancer in the subject is diagnosed, and/or a tumor type in the subject is identified. In some embodiments, the subject has pancreatic ductal adenocarcinoma (PDAC). In some embodiments, the subject is a human.

In some embodiments, the diagnosing of a cancer and/or identifying a tumor type further comprises calculating compartment weights to perform binary classification of tumor subtypes. In some embodiments, the method further comprises identifying one or more compartments within a cancer or tumor in the subject. In some embodiments, the method further comprises predicting a prognosis of an identified tumor type, and/or predicting efficacy of a treatment for an identified tumor type.

In some embodiments, the presently disclosed subject matter provides a method of estimating and/or identifying a cellular compartment in a tumor or cancer. In some embodiments the method comprises: providing a tumor or cancer tissue sample; and performing a method for de novo deconvoluting a dataset with multiple samples and/or estimating compartment weight for a single sample in accordance with the presently disclosed subject matter on the tissue sample, wherein a cellular compartment in a tumor or cancer is identified.

In some embodiments, the tumor or cancer tissue sample is from a subject, optionally from a human subject. In some embodiments, the tumor or cancer tissue sample is from any solid or hematologic malignancy and/or any liquid biopsy. In some embodiments, the tumor or cancer tissue sample comprises a pancreatic cancer.

It is thus an object of the presently disclosed subject matter to provide methods for de novo deconvoluting a dataset with multiple samples and/or estimating compartment weight for a single sample. An object of the presently disclosed subject matter having been stated hereinabove, and which is achieved in whole or in part by the presently disclosed subject matter, other objects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawings will be provided by the Office upon request and payment of the necessary fee.

FIGS. 1A through 1F present an overview of DECODER and the application of it. FIG. 1A is a flow diagram showing a utility of DECODER, which includes de novo compartment deconvolution for a data set, and single-sample compartment weight estimation for TCGA cancers. FIG. 1B is a schematic diagram showing that de novo deconvolution of DECODER aims to factorize an expression matrix A into two matrices of W (gene weights for compartments or gene weights) and H (compartment weights for samples or compartment weights). FIG. 1C is a schematic diagram showing representative steps for de novo deconvolution of DECODER. FIG. 1D is a schematic diagram showing the identification of final compartments from factors in multiple runs of K˜. FIGS. 1E and 1F show the factor trees for the Moffitt microarray data set and the TCGA PAAD data set, respectively. The resultant major compartments are colored and labelled with lead lines from the legend, with the dropped factors, minor and unstable compartments denoted by black circles. Factor ID is used for labeling.

FIGS. 2A through 2P show deconvolved compartment weights for samples in TCGA PAAD data set. FIG. 2A presents images of a representative H&E-stained PDAC tumor sections showing immune cell infiltration (arrows) in samples with high immune vs low immune weight samples separated by the median. FIG. 2B is a plot showing quantification of immune clusters by H&E staining was correlated with immune weight (two-sided Wilcoxon rank-sum test). FIGS. 2C and 2D are plots showing correlations of immune weight with the leukocyte fraction (Pearson correlation) and ESTIMATE immune score (Spearman correlation). FIGS. 2E and 2F are plots showing correlations of the sum of basal tumor and classical tumor weights, with tumor purity estimated by ABSOLUTE and methylation (Pearson correlation). FIG. 2G is a correlation of the sum of activated stroma and normal stroma weights with ESTIMATE stromal score (Spearman correlation). FIG. 2H shows associations of compartment weights with the tumor subtype calls. Heatmap shows consensus clustering of TCGA samples using 50 Moffitt tumor exemplar genes. Colored tracks show compartment weights, the ratio between basal tumor and classical tumor weights (B./C.), tumor subtypes called by B./C. (DECODER subtype), and tumor subtypes called by the clustering-based Moffitt schema (Moffitt subtype). FIG. 2I is a plot showing basal tumor and classical tumor weights are compared in Moffitt Basal-like and Classical samples (paired two-sided Wilcoxon rank-sum test). FIGS. 2J and 2K show the ratio (B./C.) and difference (B.-C.) between basal tumor and classical tumor weights are compared in Moffitt Basal-like and Classical samples (two-sided Wilcoxon rank-sum test). FIG. 2L is a plot showing the receiver-operating curve (ROC) for basal tumor weight, classical tumor weight, B./C. and B.-C. in classifying subtypes. The area under the ROC (AUC) is shown for each parameter. FIG. 2M is a plot showing threshold determination for B./C. to classify subtypes based on accuracy using Moffitt tumor subtype calls as gold standard. FIG. 2N is a plot showing threshold determination for B./C. to classify subtypes based on significance to differentiate survival. FIGS. 2O and 2P are Kaplan-Meier plots of overall survival in patients with resected PDAC for Moffitt and DECODER tumor subtypes. Patients with B./C. >=1 were categorized as Basal-like, while those with B./C. <1 as Classical (log-rank test). Box plots in b, i-k show the median (center line) and interquartile range (box), with whiskers denoting 1.5 times the interquartile range above and below the upper and lower quartile, respectively

FIGS. 3A to 3K show single-sample compartment weight estimation in COMPASS and ICGC PACA-AU RNA-seq data set. FIG. 3A is a series of plots showing endocrine, exocrine, and immune weights in the COMPASS (microdissected), ICGC (PACA-AU), and TCGA (PAAD) data sets (Kruskal-Wallis test). FIGS. 3B to 3D are a series of plots showing correlations of DECODER estimated tumor weight (the sum of basal tumor and classical tumor weights), immune weight, and stroma weight (the sum of activated stroma and normal stroma weights) with ABSOLUTE tumor purity (Pearson correlation), ESTIMATE immune score (Spearman correlation), and ESTIMATE stromal score (Spearman correlation), respectively. FIG. 3E is a series of plots showing exocrine, immune, classical tumor, and basal tumor weights for the ICGC-subtyped ADEX, Immunogenic, Pancreatic Progenitor, and Squamous samples in the ICGC PACA-AU RNA-seq data set (Kruskal-Wallis test). FIGS. 3F and 3G show associations of compartment weights with the tumor subtype calls. Colored tracks show compartment weights, the ratio between basal tumor and classical tumor weights (B./C.), tumor subtypes called by B./C. (DECODER subtype), tumor subtypes called by the clustering-based Moffitt schema (Moffitt subtype) and subtypes called by ICGC (ICGC subtype). For COMPASS, samples are ordered by consensus clustering using 50 Moffitt tumor exemplar genes. For ICGC, samples are ordered by ICGC subtypes. FIG. 3H is a plot showing B./C. for ICGC samples grouped by intraductal papillary mucinous neoplasm (IPMN), Classical PDAC, Basal-like PDAC and adenosquamous carcinoma (Kruskal-Wallis test). FIG. 3I is a Kaplan-Meier plot of DECODER subtypes in the ICGC data set (Log-rank test). Patients with B./C. >=1 were categorized as Basal-like, while those with B./C. <1 as Classical. FIGS. 3J and 3K are plots showing correlations of the percent change (% change) in size of tumor target lesions from baseline, with B./C. in DECODER Basal-like and Classical samples (Pearson correlation). One Basal-like sample with an unstable DNA subtype was removed. Treatment of gemcitabine plus nab-paclitaxel (GP) and modified-FOLFIRINOX (FFX) was colored, respectively. Box plots in FIGS. 3A, 3E, and 3H show the median (center line) and interquartile range (box), with whiskers denoting 1.5 times the interquartile range above and below the upper and lower quartile, respectively

FIGS. 4A through 4D show de novo deconvolution on TCGA ATAC-seq PanCan data set. FIG. 4A shows unsupervised hierarchical clustering on samples (columns) using compartment weights on the heatmap. The darker the color, the higher the weight for the respective compartment and sample. Tracks above show cancer types, ATAC-seq-based cluster calls, and iCluster calls for the samples. Compartments (rows on the heat map) were manually ordered so that the clusters of enriched weights are shown on the diagonal. The same order of the compartments is maintained in FIGS. 4B-4D. The name of each compartment is generated by concatenating an uppercase letter “D”, factor ID, a colon (:) and the manual annotation for the compartment. FIG. 4B shows compartment weights for each cancer type. Box plots show the median (center line) and interquartile range (box), with whiskers denoting 1.5 times the interquartile range above and below the upper and lower quartile, respectively. FIGS. 4C and 4D show associations of samples weights (rows) with ATAC-seq-based clusters and iClusters (columns). In each ATAC-seq-based cluster or iCluster, the means of the compartment weights are shown on the heatmap. Columns on the heat maps were manually ordered so that the best match with DECODER compartments are shown on the diagonal.

FIGS. 5A-5C show compartments identified by the de novo deconvolution of DECODER in the Moffitt microarray dataset. FIG. 5A shows a correlation of gene weights for compartments derived by DEOCDER with gene weights for factors identified in the previous study using empirical number of factors (K=14). FIG. 5B shows a number of overlaps in top 250 genes for compartments derived by DECODER and by the previous study. FIG. 5C shows an association of sample weights to known tissue labels. Sample weights derived by DECODER are shown as grayscale bars. Solid color bars show the tissue of origin and tumor status of the samples, which were used to order the samples horizontally. All tumors, cell lines and adjacent normal tissues in the microarray dataset are shown.

FIGS. 6A to 6B2 show an association of compartments across 33 TCGA cancer types. FIG. 6A shows percentages of overlaps in top 250 genes (converted to decimals) for 269 compartments shown on heatmap. Compartments were clustered by euclidean distance indicating similarity. Clusters of immune, activated stroma, histone and basal tumor compartments across cancer types are shown as row tracks. FIGS. 6B1 and 6B2 show percentages of overlaps in top 250 genes (converted to decimals) for closely resembled compartments across cancer types.

FIGS. 7A through 7F show marker genes for compartments in TCGA PAAD. FIG. 7A shows identification of marker genes for each compartment. FIGS. 7B and 7C show correlations of the immune fraction calculated using marker genes for the immune compartment, with the leukocyte fraction and ESTIMATE immune score. FIGS. 7D and 7E show correlations of the tumor fraction (sum of basal and classical fraction) with the tumor fraction estimated by ABSOLUTE and methylation. FIG. 7F shows correlation of the stroma fraction (sum of activated and normal fraction) with the ESTIMATE stromal score.

FIGS. 8A1 to 8A6 are Kaplan-Meier plots of patient outcome stratified by immune weights. Overall survival (OS) were analyzed for each cancer type separately where the immune compartment was available. Immune-high and immune-low patients were classified by the median of the immune weight in each cancer type separately. Log-rank test was used to derive the p-value.

FIGS. 9A to 9F show single-sample compartment weight estimation. FIGS. 9A and 9B are plots showing ten-fold cross validation comparing sample weights derived from de novo deconvolution, and sample weights estimated using trained gene weights by the non-negative least square (NNLS) algorithm. FIGS. 9C and 9E are plots showing an association of compartment weights for samples with the Moffitt tumor subtype calls in the COMPASS dataset. FIGS. 9D and 9F are plots showing an association of compartment weights for samples with the Moffitt tumor subtype calls in the ICGC dataset. Two-sided Wilcoxon rank-sum test was used to derive the p-value.

### DETAILED DESCRIPTION

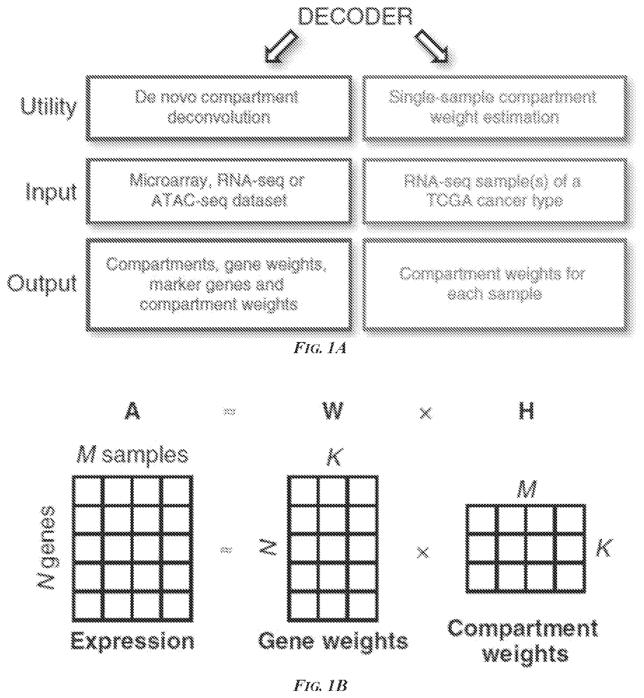

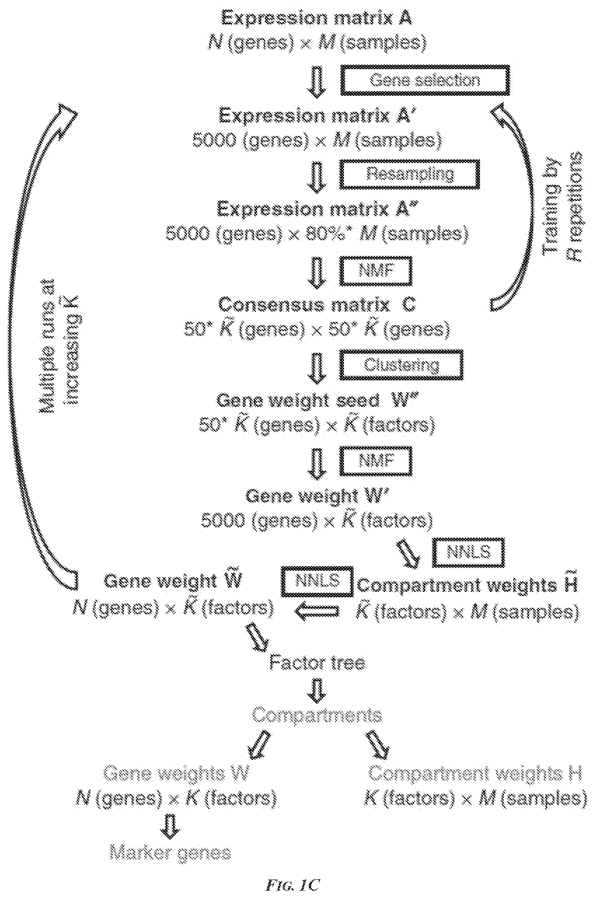

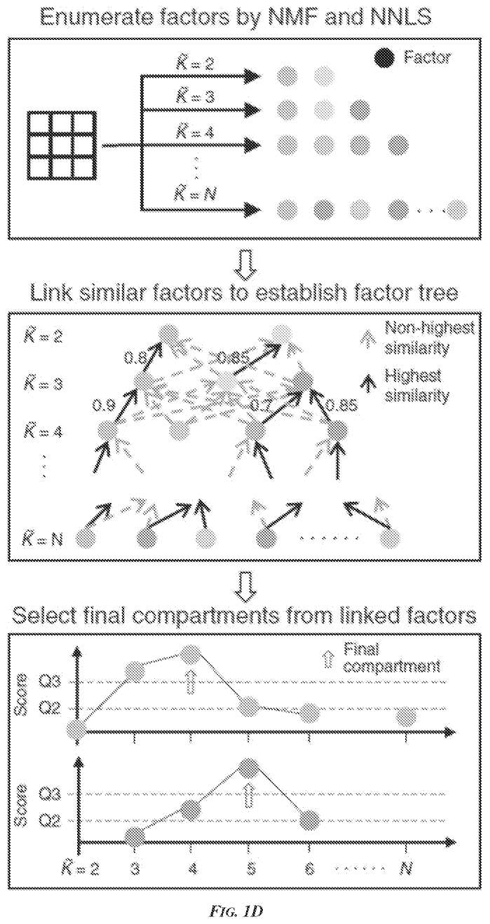

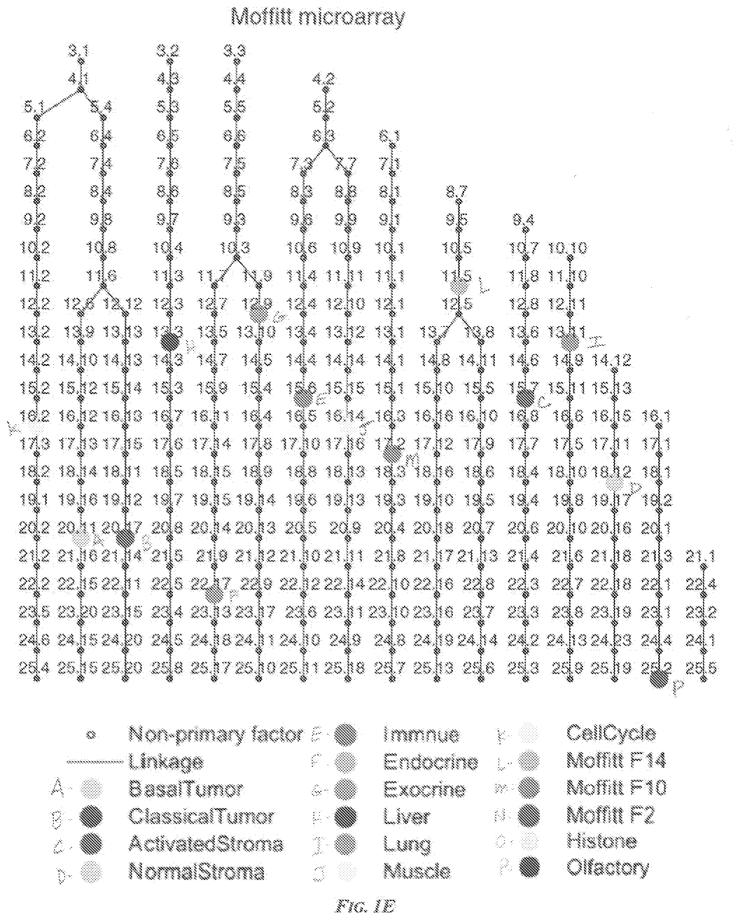

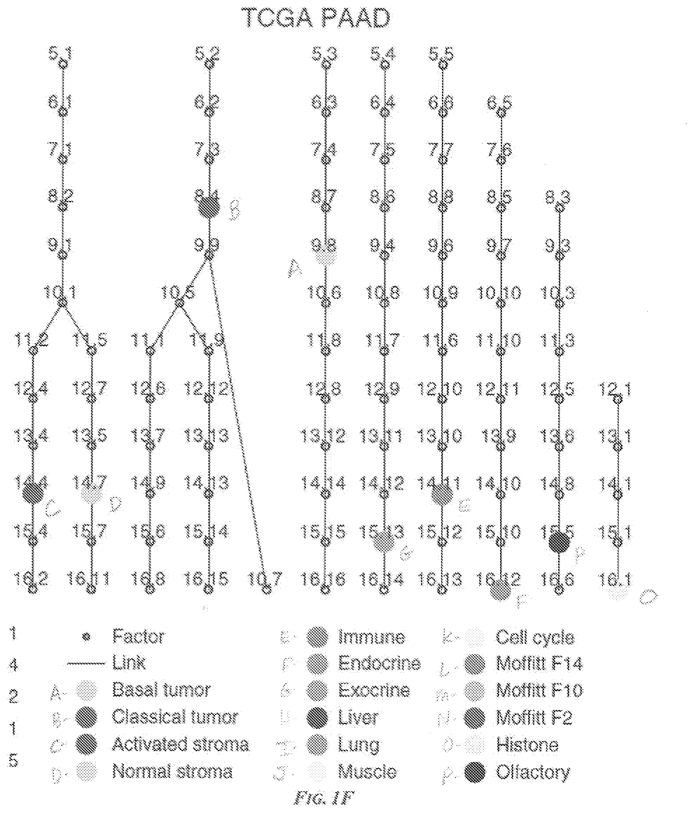

Disclosed herein is de novo compartment deconvolution and weight estimation of tumor samples (DECODER), an NMF-based integrated and sophisticated framework for the de novo deconvolution of tumor mixture samples for compartments, and estimation of compartment weights for samples (FIG. 1A). It is shown that DECODER can perform automated de novo deconvolution for patient samples to derive dynamic and biologically sound compartments without setting ad hoc parameters. By applying DECODER to RNA-sequencing (RNA-seq) data from 33 The Cancer Genome Atlas (TCGA) cancer types, 269 cancer-specific compartments are identified, with a list of marker genes for each compartment. In addition, DECODER can be used to calculate the compartment weights for a single sample, making it applicable in the clinical setting. By applying DECODER to PDAC microarray (Moffitt et al., 2015), TCGA pancreatic adenocarcinoma (PAAD; The Cancer Genome Atlas Research Network, 2017), COMPASS (Aung et al., 2018), and ICGC (International Cancer Genome Consortium) pancreatic cancer (Bailey et al., 2016) RNA-seq data sets, it is demonstrated herein that DECODER provides insight into pancreatic cancer biology with clinical implications. This framework is not only a useful algorithm for de novo and single-sample deconvolution of heterogenous samples, but also, for the first time, provides cancer type-specific derived compartment information.

Definitions

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a,” “an,” and “the” mean “one or more” when used in this application, including the claims. Thus, the phrase “a cell” refers to one or more cells, unless the context clearly indicates otherwise.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The term “comprising,” which is synonymous with “including,” “containing,” and “characterized by,” is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, and/or ingredient not specifically recited. For example, when the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter.

With respect to the terms “comprising,” “consisting essentially of,” and “consisting of,” where one of these three terms is used herein, the presently disclosed and claimed subject matter can include the use of either of the other two terms. For example, it is understood that the methods of the presently disclosed subject matter in some embodiments comprise the steps that are disclosed herein and/or that are recited in the claims, in some embodiments consist essentially of the steps that are disclosed herein and/or that are recited in the claims, and in some embodiments consist of the steps that are disclosed herein and/or that are recited in the claim.

The term “subject” as used herein refers to a member of any invertebrate or vertebrate species. Accordingly, the term “subject” is intended to encompass any member of the Kingdom Animalia including, but not limited to the phylum Chordata (i.e., members of Classes Osteichythyes (bony fish), Amphibia (amphibians), Reptilia (reptiles), Ayes (birds), and Mammalia (mammals)), and all Orders and Families encompassed therein. In some embodiments, the presently disclosed subject matter relates to human subjects.

Similarly, all genes, gene names, and gene products disclosed herein are intended to correspond to orthologs from any species for which the compositions and methods disclosed herein are applicable. Thus, the terms include, but are not limited to genes and gene products from humans. It is understood that when a gene or gene product from a particular species is disclosed, this disclosure is intended to be exemplary only, and is not to be interpreted as a limitation unless the context in which it appears clearly indicates. Thus, for example, the genes and/or gene products disclosed herein are also intended to encompass homologous genes and gene products from other animals including, but not limited to other mammals, fish, amphibians, reptiles, and birds.

The methods and compositions of the presently disclosed subject matter are particularly useful for warm-blooded vertebrates. Thus, the presently disclosed subject matter concerns mammals and birds. More particularly provided is the use of the methods and compositions of the presently disclosed subject matter on mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the application of the methods and compositions of the presently disclosed subject matter to livestock, including but not limited to domesticated swine (pigs and hogs), ruminants, horses, poultry, and the like.

The term “about,” as used herein when referring to a measurable value such as an amount of weight, time, dose, etc., is meant to encompass variations of in some embodiments ±20%, in some embodiments ±10%, in some embodiments ±5%, in some embodiments ±1%, and in some embodiments ±0.1% from the specified amount, as such variations are appropriate to perform the disclosed methods and/or to employ the presently disclosed arrays.

As used herein the term “gene” refers to a hereditary unit including a sequence of DNA that occupies a specific location on a chromosome and that contains the genetic instruction for a particular characteristic or trait in an organism. Similarly, the phrase “gene product” refers to biological molecules that are the transcription and/or translation products of genes. Exemplary gene products include, but are not limited to mRNAs and polypeptides that result from translation of mRNAs. Any of these naturally occurring gene products can also be manipulated in vivo or in vitro using well known techniques, and the manipulated derivatives can also be gene products. For example, a cDNA is an enzymatically produced derivative of an RNA molecule (e.g., an mRNA), and a cDNA is considered a gene product. Additionally, polypeptide translation products of mRNAs can be enzymatically fragmented using techniques well known to those of skill in the art, and these peptide fragments are also considered gene products.

The term “isolated,” as used in the context of a nucleic acid or polypeptide (including, for example, a nucleotide sequence, a polypeptide, and/or a peptide), indicates that the nucleic acid or polypeptide exists apart from its native environment. An isolated nucleic acid or polypeptide can exist in a purified form or can exist in a non-native environment.

Further, as used for example in the context of a cell, nucleic acid, polypeptide, or peptide, the term “isolated” indicates that the cell, nucleic acid, polypeptide, or peptide exists apart from its native environment. In some embodiments, “isolated” refers to a physical isolation, meaning that the cell, nucleic acid, polypeptide, or peptide has been removed from its native environment (e.g., from a subject).

The terms “nucleic acid molecule” and “nucleic acid” refer to deoxyribonucleotides, ribonucleotides, and polymers thereof, in single-stranded or double-stranded form. Unless specifically limited, the term encompasses nucleic acids containing known analogues of natural nucleotides that have similar properties as the reference natural nucleic acid. The terms “nucleic acid molecule” and “nucleic acid” can also be used in place of “gene,” “cDNA,” and “mRNA.” Nucleic acids can be synthesized, or can be derived from any biological source, including any organism.

As used herein, the terms “peptide” and “polypeptide” refer to polymers of at least two amino acids linked by peptide bonds. Typically, “peptides” are shorter than “polypeptides,” but unless the context specifically requires, these terms are used interchangeably herein.

As used herein, a cell, nucleic acid, or peptide exists in a “purified form” when it has been isolated away from some, most, or all components that are present in its native environment, but also when the proportion of that cell, nucleic acid, or peptide in a preparation is greater than would be found in its native environment. As such, “purified” can refer to cells, nucleic acids, and peptides that are free of all components with which they are naturally found in a subject, or are free from just a proportion thereof.

### Exemplary Embodiments

In some embodiments, the presently disclosed subject matter provides a method for de novo deconvoluting a dataset with multiple samples and/or estimating compartment weight for a single sample. In some embodiments, the method comprising applying the following steps to a dataset with multiple samples or to a single sample: using a nonnegative matrix factorization (NMF) algorithm to calculate a NMF seed training configured to calculate a stable gene weight seed (W″), wherein W″ is a 50*×\tilde{K} matrix of 50*\tilde{K} rows of genes and k columns of factor, completed for R repetitions resulting in R data partitions; executing an NMF algorithm for each of R data partitions; calculating a gene weight matrix for a current number (K) of factors; and executing a final NMF algorithm to generate a gene weight matrix W′ (a 5000*\tilde{K} matrix of 5000 rows of genes and \tilde{K} columns of factors), and compartment weight matrix H′ (a \tilde{K}×M matrix of \tilde{K} rows of factors and M columns of samples), whereby the dataset is deconvoluted and/or a compartment weight is estimated.

In some embodiments, the R repetitions is about 10,000 or greater. In some embodiments, the NMF algorithm for each of the R data partitions comprises an unsupervised NMF executed with at least 20 randomly initialized instances of NMF using a multiplicative update NMF solver for ten steps using a built-in NMF function in MATLAB (R2017b).

In some embodiments, the method further comprises calculating a consensus matrix, wherein the consensus matrix represents a frequency of the genes to be determined as the top genes, and wherein the consensus matrix is used for hierarchical clustering to yield K gene clusters. In some embodiments, the calculation of the gene weight matrix comprises ranking genes in a matrix in descending order of a weight difference between a current factor weight and a largest weight in the rest of the factors, wherein a top 50 genes for any factor are recorded in a gene-by-gene consensus matrix C (50*\tilde{K}×50*\tilde{K}).

In some embodiments, the method comprises using a non-negative least square (NNLS) algorithm to find:

arg minₕ_{ᵢ}∥W′\tilde{h}ᵢ−a′ᵢ∥₂subject to \tilde{h}ᵢ≥0,

where \tilde{h}ᵢ is the ith column/sample in \tilde{H} (a \tilde{K}×M matrix) to be determined, a′ᵢ is the ith column/sample in A′ (a 5000×M matrix, wherein \tilde{H} is considered to record the final compartment weights for each factor at the current number of factors (\tilde{K}) from the de novo deconvolution.

In some embodiments, the method further comprises using a NNLS algorithm to find:

arg min_{w}_{ᵢ}∥\tilde{H}\tilde{w}ⱼ−aⱼ∥₂subject to \tilde{W}ⱼ≥0,

where \tilde{w}ⱼ is the jth row/gene in \tilde{W} (a N×\tilde{K} matrix) to be determined, and aⱼ is the jth row/gene in A (a N×M matrix), wherein \tilde{W} is considered to record the final gene weights for each factor at the current number of factors (\tilde{K}) from the de novo deconvolution, wherein gene weight matrix (\tilde{W}) is further used for ranking of genes, calculation of factor scores, and/or annotation of compartments.

In some embodiments, the dataset is from any platform. In some embodiments, the dataset comprises RNA sequence and/or gene expression data. In some embodiments, the RNA sequence and/or gene expression data is from a tumor, optionally ATACseq. In some embodiments, the tumor is any tumor, such as but not limited to a pancreatic cancer, a prostate cancer, a bladder cancer, or a breast cancer. In some embodiments, the tumor is from a subject, optionally a human subject. In some embodiments, the dataset is derived from any one of Tables 1-6, or any combination thereof. In some embodiments, the dataset is derived from Data Table 1.

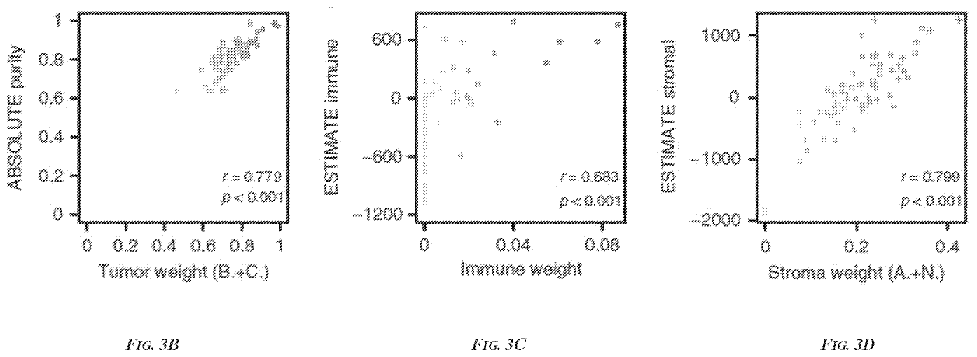

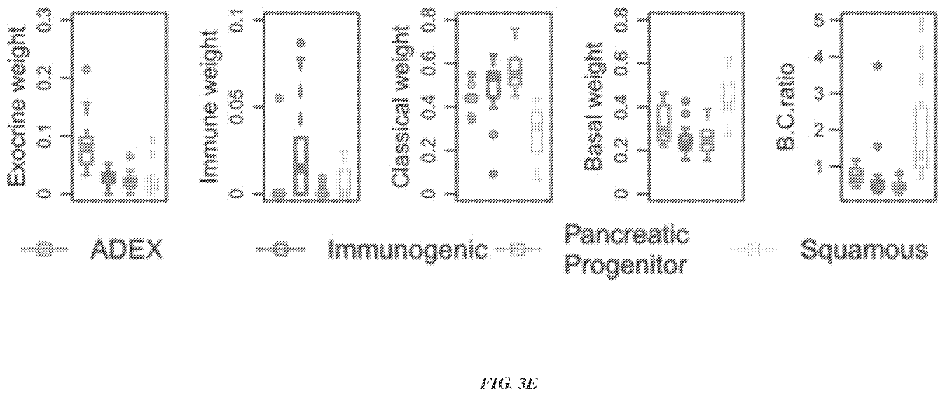

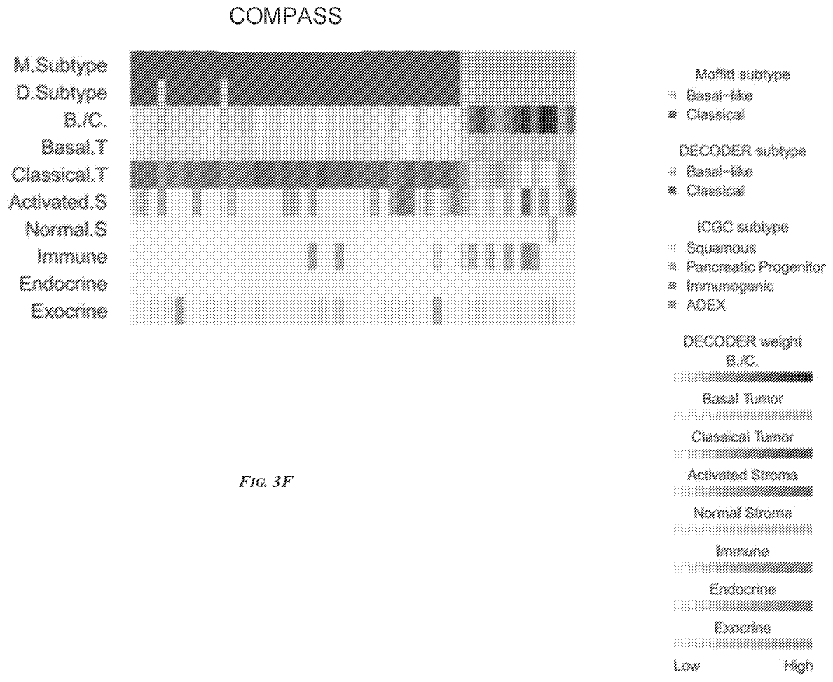

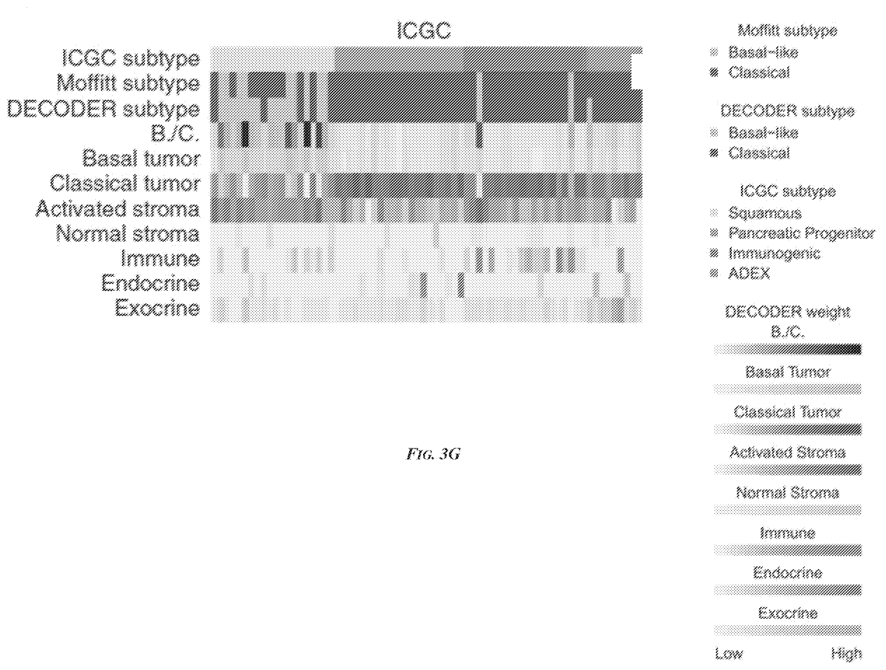

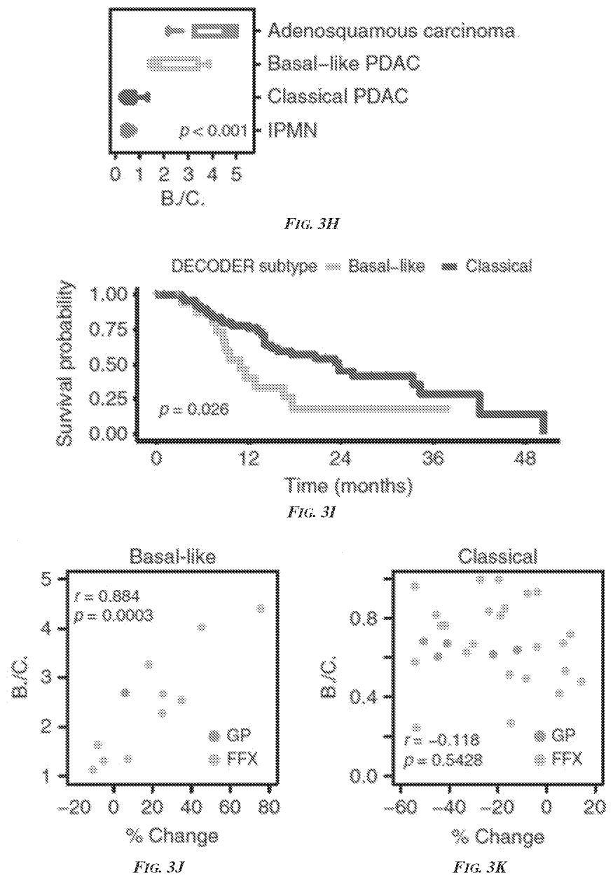

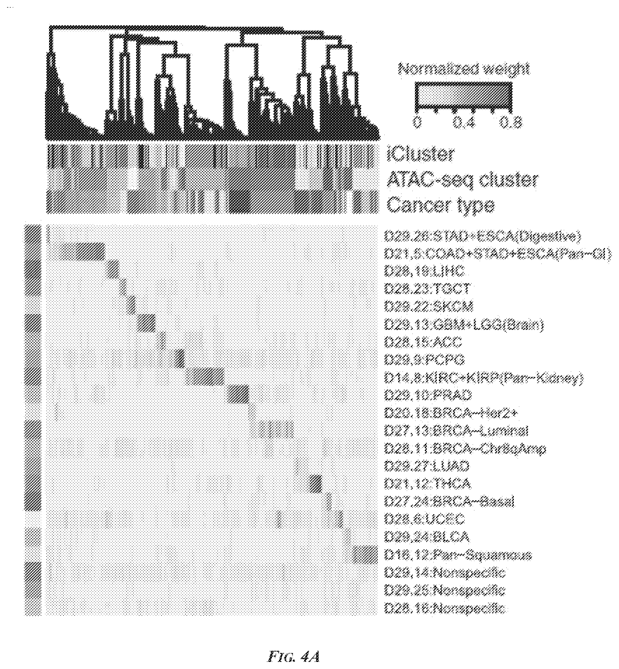

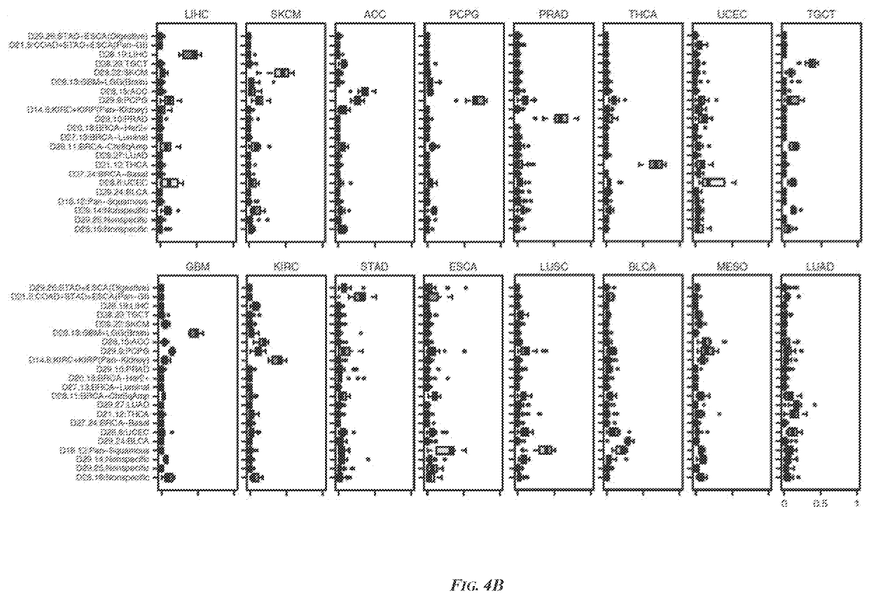

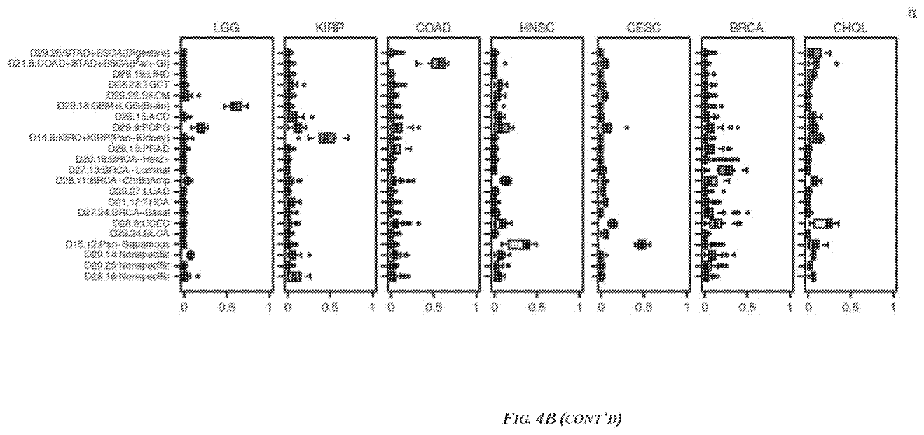

In some embodiments, the presently disclosed subject matter provides a method of diagnosing a cancer and/or identifying a tumor type in a subject. In some embodiments, the method comprises: providing a sample from a subject to be diagnosed; and performing a method for de novo deconvoluting a dataset with multiple samples and/or estimating compartment weight for a single sample in accordance with the presently disclosed subject matter on the sample from the subject, wherein a cancer in the subject is diagnosed, and/or a tumor type in the subject is identified. In some embodiments, the subject has pancreatic ductal adenocarcinoma (PDAC). In some embodiments, the subject is a human. For pancreatic cancer it is shown in FIG. 3I that the compartments weights predict prognosis in pancreatic cancer. In FIG. 3J the compartment weights are associated with treatment response to FFX (FOLFIRINOX) and GP (Gemcitabine plus nab-paclitaxel). Therefore, the compartment weight can be used to select treatment, for example patients that would respond, or not respond to FFX.

In some embodiments, the diagnosing of a cancer and/or identifying a tumor type further comprises calculating compartment weights to perform binary classification of tumor subtypes. In some embodiments, the method further comprises identifying one or more compartments within a cancer or tumor in the subject. In some embodiments, the method further comprising predicting a prognosis of an identified tumor type, and/or predicting efficacy of a treatment for an identified tumor type.

In some embodiments, the presently disclosed subject matter provides a method of estimating and/or identifying a cellular compartment in a tumor or cancer. In some embodiments the method comprises: providing a tumor or cancer tissue sample; and performing a method for de novo deconvoluting a dataset with multiple samples and/or estimating compartment weight for a single sample in accordance with the presently disclosed subject matter on the tissue sample, wherein a cellular compartment in a tumor or cancer is identified.

In some embodiments, the tumor or cancer tissue sample is from a subject, optionally from a human subject. In some embodiments, the tumor or cancer tissue sample is from any solid or hematologic malignancy and/or any liquid biopsy. In some embodiments, the tumor or cancer tissue sample comprises a pancreatic cancer.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

### Materials and Methods for the EXAMPLES

NMF seed training. To handle the stochastic nature of the NMF, a stable gene weight seed (W″) was trained before the application of a final NMF (FIG. 1C). W″ is a 50*×\tilde{K} matrix of 50*\tilde{K} rows of genes and \tilde{K} columns of factors. For de novo deconvolution of microarray and RNA-seq expression profiles recorded as A (a N×M matrix of N genes and M samples), highly expressed genes in the top third quartile were subjected to selection of the 5000 most variable ones resulting in A′ (a 5000×M matrix). R (R=10,000 by default and in this study) repetitions of fivefold resampling (˜80% of the samples, A″) were performed. For each of the R data partitions, unsupervised NMF was executed with 20 randomly initialized instances of NMF using the multiplicative update NMF solver for ten steps using the built-in NMF function in MATLAB (R2017b). The pair of gene weights and compartment weights with the lowest residual solution from these 20 instances were then used to seed NMF of A″ to convergence with the alternating least-squares solver. The result contains a gene weight matrix for the current number (K) of factors (genes as rows and factors as columns). Based on this gene weight matrix, for each factor (column), the genes were ranked in descending order of the weight difference between the current factor weight and the largest weight in the rest of the factors. The top 50 genes for any factor were then recorded in a gene-by-gene consensus matrix C (50*\tilde{K}×50*\tilde{K}) by approximation due to possible duplicated top genes in multiple factors). This consensus matrix represents the frequency of the genes to be determined as the top genes, and was then used for hierarchical clustering to yield \tilde{K} gene clusters. These K gene clusters were used to create a seed matrix W″, so that the top genes for the respective cluster have loading value of 1, with the rest of the genes having loading value of 0.01. This gene weights seed W″ was then used to seed a final NMF, with a robust deconvolution of gene weights (W′) and compartment weights (H′). Of note, none of A′, A″ W′, W″, H′, or H″ involves matrix transpose for A, W, or H; instead, they represent related but different matrices. Similarly, 8000 most highly expressed and variable ATAC-seq peaks were involved in the seed training process.

Compartment weights and gene weights projection. The final NMF outputs two matrices, i.e., gene weight matrix W′ (a 5000*\tilde{K} matrix of 5000 rows of genes and \tilde{K} columns of factors), and compartment weight matrix H′ (a \tilde{K}×M matrix of \tilde{K} rows of factors and M columns of samples). Subsequently, NNLS was used to find:

arg minₕ_{ᵢ}∥W′\tilde{h}ᵢ−a′ᵢ∥₂subject to \tilde{h}ᵢ≥0 (1)
where \tilde{h}ᵢ is the ith column/sample in \tilde{H} (a \tilde{K}×M matrix) to be determined, a′ᵢ is the ith column/sample in A′ (a 5000×M matrix). Of note, while discarding H′, \tilde{H} is considered to record the final compartment weights for each factor at the current number of factors (\tilde{K}) from the de novo deconvolution.

With \tilde{H} derived by estimating each \tilde{h}ᵢ, similarly, NNLS was used to find

arg min_{w}_{ⱼ}∥\tilde{H}\tilde{w}ⱼ−aⱼ∥₂subject to \tilde{W}ⱼ≥0 (2)
where \tilde{w}ⱼ is the jth row/gene in \tilde{W} (a N×\tilde{K} matrix) to be determined, and aⱼ is the jth row/gene in A (a N×M matrix). As a result, \tilde{W} is considered to record the final gene weights for each factor at the current number of factors (\tilde{K}) from the de novo deconvolution. This gene weight matrix (\tilde{W}) is further used for ranking of genes, calculation of factor scores, and annotation of compartments.

Minimum and maximum of \tilde{K} to be considered. The range of increasing \tilde{K} can be customized by the user. By default, the step of factor deconvolution starts with \tilde{K}=2 and ends when the median of factor scores are <0.5 for more than three consecutive runs. In addition, starting from \tilde{K}=2, the run at \tilde{K} will begin to be considered until the median of the factor scores at \tilde{K} are >0.5.

Factor score calculation and linkage establishment. The factor ID is generated by concatenating the number of factors at current run (\tilde{K}), a dot (•), and an unduplicated number from 1 to \tilde{K} randomly assigned by the algorithm. To score each factor, genes for each factor at each run of \tilde{K} are ranked to determine the top genes. Specifically, for a factor (ith column in ˜W) at each run of K., the genes were ranked in descending order of the weight differences between the loading value in the ith column and the largest loading value in the rest of the columns. Based on the ranking, the top 250 genes are identified. The overlapping percentages converted to decimals of the top 250 genes between each of the factors at \tilde{K} and each of the factors at \tilde{K}−1 (similarity) were calculated. For the ith factor at \tilde{K}, if the iith factor at \tilde{K}−1 showed the largest overlap (highest similarity) and was greater than 0.1, then the overlapping percentage converted to decimal (similarity) is determined as the score for the ith factor at \tilde{K}. If the two factors ith at \tilde{K} and iith at \tilde{K}−1 were considered to be associated (i.e., largest overlapping percentage and greater than 0.1), then a link is established between them (FIG. 1D). However, if the similarity between the ith factor at K and the factors at \tilde{K}−1 was <0.1, the ith factor at K will be considered a newly emerged factor. If there is no association between the ith factor at \tilde{K}, and any of the factors at \tilde{K}+1, then no linkage is established as the respective factor was considered too split or diluted to be detected in the resolution of \tilde{K}+1. In this way, linkages of associated factors in each run of a different are established.

Compartments identification from factors. The linking of all the related factors at multiple runs of \tilde{K} leads to the placement of all the identified factors on a tree-like plot (FIGS. 1E and 1F). Along each linkage, the pattern of the factor scores is examined to determine the optimal factor along each linkage. We assume that the consistently relative high scores of adjacently linked factors indicate the existence of a stable biological component. Therefore, compartment identification was performed, so that a resultant compartment: (a) is on a linkage with >2 factors on it, (b) shows a score greater than the third quartile (Q₃) of all the scores, (c) has more than two continuous (adjacently linked) (major) or one (unstable) factor(s) greater than the median quartile (Q₂) of all the scores, (d) is with the maximum score among the continuous factors with score higher than Q₂ (candidates in a high-score factor block), and (e) has the largest number of factors in the high-score block or has the greatest score if multiple high-score blocks and candidates found. Finally, if a major compartment is found to have over 100 overlapping genes in the top 250 with another major compartment, and both of them have the same linkage, the compartment identified at larger \tilde{K} is then labeled as a “minor” compartment. For each of the final compartments, respective columns of gene weights are extracted from \tilde{W} of the respective run at K, and combined to form a final gene weights matrix W. W is saved as a reference for later single-sample compartment weight estimation. Similarly, rows of \tilde{H} corresponding to the resultant compartments are extracted to from the final compartment weights H for the samples in the bulk measurement A.

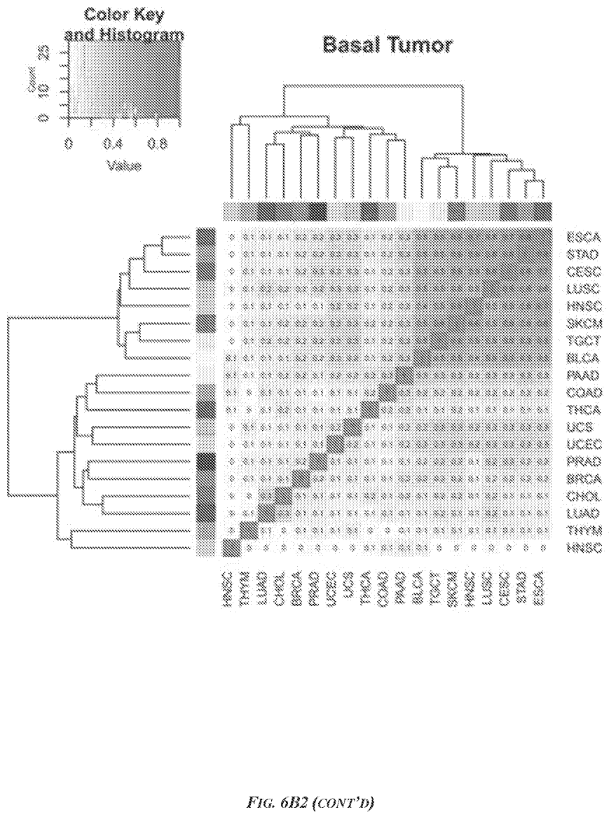

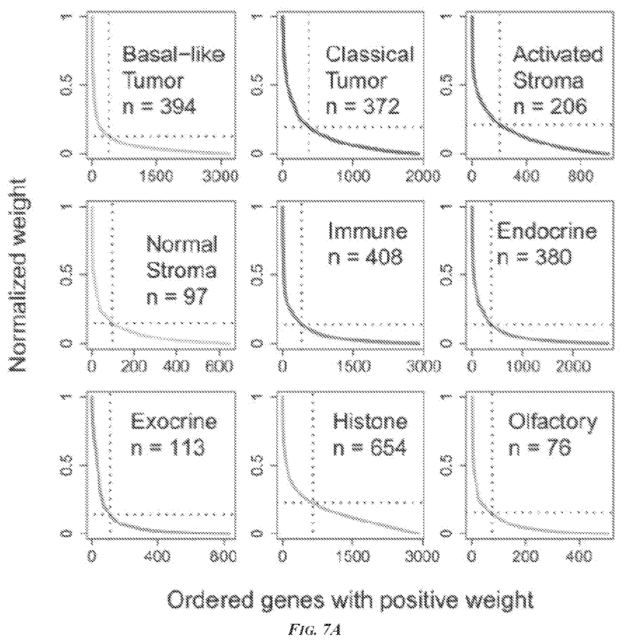

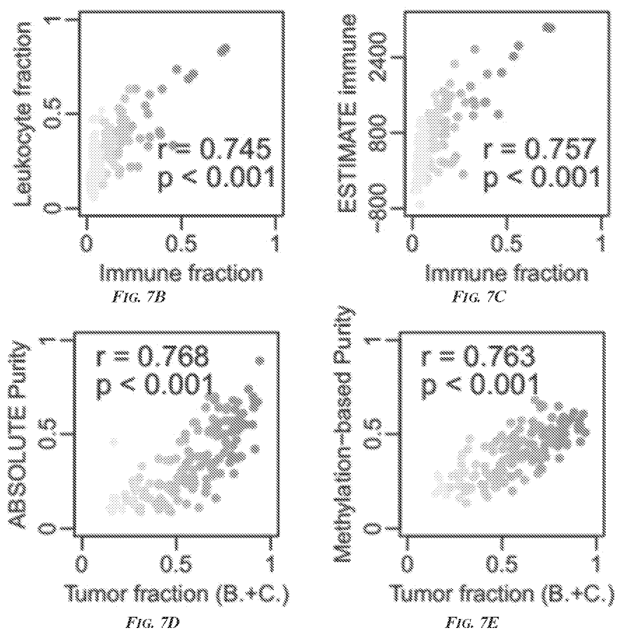

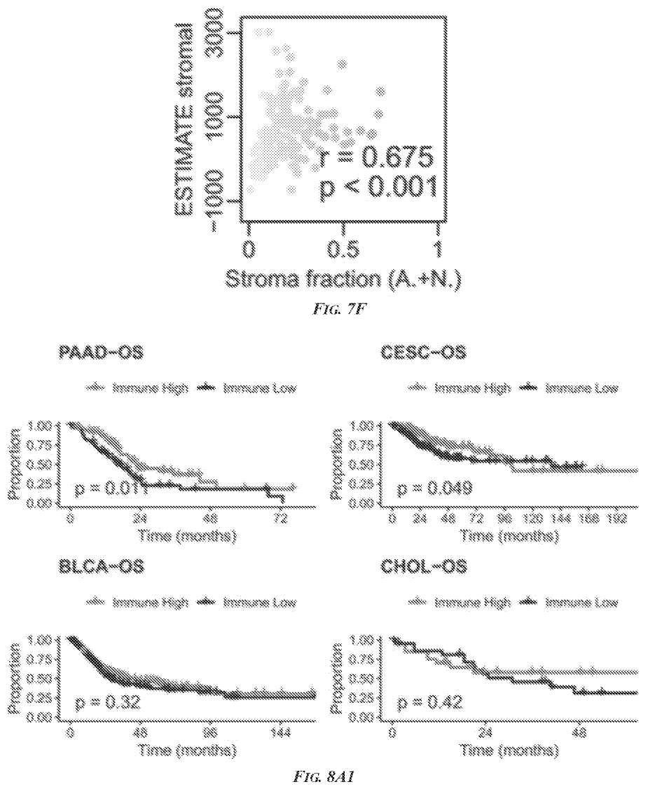

Gene analysis and compartment annotation. For each compartment, the marker genes were identified as the top genes with positive normalized weight differences exponentially greater than the rest, which are above the geometric inflection point on the ranking curve (FIG. 7A). Ranked genes in each compartment were subjected to gene set analysis using annotated gene sets from MSigDB v3.1 (Martens et al., 2019), assessed by the Kolmogorov-Smirnov statistic with Benjamini-Hochberg correction for significance (Moffitt et al., 2015). For RNA-seq data, final compartments of TCGA PAAD were annotated based on MSigDB terms and prior knowledge. For the remaining cancer types, the immune, activated stroma, basal tumor, olfactory, and histone compartments were annotated according to MSigDB, with the remaining compartments unannotated.

Single-sample compartment weight estimation. Given a new bulk measurement B, which is a N×M matrix of N rows of genes and M columns of samples, and precomputed gene weights W derived from de novo deconvolution, which is a N×K matrix of N rows of genes and K columns of compartments, NNLS is used to find

arg mini_{\tilde{h}}_{ᵢ}∥Whᵢ−bᵢ∥₂subject to hᵢ≥0 (3)
where hᵢ is the ith column/sample in H to be determined, bᵢ is the ith column/sample in B.

Compartment weight normalization. For pancreatic cancer, weights for seven compartments (basal tumor, classical tumor, activated stroma, normal stroma, immune, endocrine, and exocrine) were normalized so that the sum of them equals to 1 for each sample. For PanCan ATAC-seq data set, weights for the 22 major compartments were normalized so that the sum of them equals to 1 for each sample.

Ten-fold cross-validation. In TCGA PAAD, samples were randomly partitioned into ten subsets. De novo deconvolution was then performed on each combination of nine-fold of samples to derive final gene weights. Then for each onefold of samples, the compartment weights were then estimated by the gene weights derived by the other nine-fold of samples by the non-negative least square (NNLS) algorithm. Finally, the estimated compartment weights were compared with those derived from performing de novo deconvolution on the whole data set.

Statistical information. Two-sided paired Wilcoxon rank-sum test was used for comparison of basal and classical tumor weights in subtyped samples. Two-sided Wilcoxon rank-sum test was used to compare ratios and differences of basal and classical tumor weights in subtyped samples. The Kruskal-Wallis test by ranks was used for testing whether the compartment weights originated from the same distribution when more than two categories of samples were involved. In correlation analysis, Spearman correlation was used to compare DECODER-derived weights with ESTIMATE-derived scores, where the scales were largely different. Otherwise, Pearson correlation was involved. For survival analysis, continuous variables were analyzed by Cox proportional-hazards regression model, and categorical variables were analyzed by log-rank test.

Data availability. A published collection of microarray data (GSE71729 and GSE21501) from a previous study (Moffitt et al., 2015) was used for comparison between de novo deconvolution of DECODER and previous NMF run in the study. TCGA normalized RNA-seq gene expression data from 33 cancer types were downloaded from the Broad Institute FIREHOSE portal [http://gdac.broadinstitute.org]. For the PAAD data set, out of 183 samples deposited in the portal, we included 150 white-listed PDAC samples for further analysis (The Cancer Genome Atlas Research Network, 2017). For the rest of the cancer types, all of the downloaded samples were used for the de novo deconvolution analysis. The normalized ATAC-seq counts within the pan-cancer peak set were downloaded from the Genomic Data Commons web site [https://gdc.cancer.gov/aboutdata/publications/ATACseq-AWG]. Loci with mean counts across samples greater than the overall mean, and standard deviation (SD) of counts across samples greater than the mean of all SD (n=105,138) were subjected to de novo deconvolution of DECODER. Seventy samples with matched mRNA subtype calls from Bailey et al., 2016, and RNA-seq data, as well as clinical data (PACA-AU) were downloaded from ICGC data portal [http://dcc.icgc.org/]. RNA-seq data of 50 PDAC samples that underwent laser capture microdissection in COMPASS trial (Aung et al., 2018) were obtained from European Genome-Phenome Archive (EGA, EGAS00001002543) with granted access. A reporting summary for this data is provided in Table 1 set forth below.

Code availability. A Matlab repository for DECODER is available at GitHub [https://github.com/laurapeng/decoder], with the utilities of de novo deconvolution and single sample compartment weight estimation. MATLAB R2017b is recommended for use. All the downloaded data sets are also deposited in this repository, along with all the deconvolution results in this study. The configure files for de novo deconvolution (Moffitt microarray, TCGA RNAseq, and TCGA PanCan ATAC-seq data sets), and single-sample compartment weight estimation (COMPASS PDAC and ICGC pancreatic cancer data sets) are provided as Tables 2-6 set forth below. In addition, an R package for single sample compartment weight estimation for TCGA cancers is readily accessible at GitHub [https://github.com/laurapeng/decoder]. R version 3.4 is recommended for use.

TABLE 1
Datasets involved in this study

| Dataset | Description | Link |
|---|---|---|
| Moffitt | Primary tumor, | https://www.ncbi.nlm.nih.gov/geo/ |
| Microarray | metastatic and | query/acc.cgi?acc=GSE71729 |
| | normal samples | https://www.ncbi.nlm.nih.gov/geo/ |
| | | query/acc.cgi?acc=GSE21501 |
| TCGA RNA-seq | 33 cancer types | http://gdac.broadinstitute.org |
| 33 cancer types | | |
| COMPASS trial | Microdissected, | https://www.ebi.ac.uk/ega/ |
| | treatment response | studies/EGAS00001002543 |
| | available | |
| ICGC PACA- | Panceatic cancers | http://dcc.icgc.org/ |
| AU | including PDAC, | |
| | IPMN, | |
| | adenoquamous | |
| | carcinomas | |
| | and acinar | |
| | cell carcinomas | |
| TCGA ATAC- | 23 cancer types | https://gdc.cancer.gov/aboutdata/ |
| seq | | publications/ATACseq-AWG |

TABLE 2
Configure file for de novo deconvolution of
the Moffitt microarray dataset

| dataType | Microarray |
|---|---|
| dataMatrix | Moffitt_PDAC_array.mat |
| dataFormat | mat |
| geneIDType | geneSymbol |
| logTransformed | yes |
| rangeK | 2:25 |
| repTimes | 10000 |

TABLE 3
Configure file for de novo deconvolution of the
TCGA RNA-seq datasets.
TCGA pancreatic adenocarcinoma (PAAD) RNA-
seq data is provided as demo_data.tsv.

| dataType | RNAseq |
|---|---|
| dataMatrix | demo_data.tsv |
| dataFormat | tsv |
| geneIDType | TCGA |
| logTransformed | no |
| rangeK | auto |
| repTimes | 10000 |

TABLE 4
Configure file for de novo deconvolution of the
TCGA ATAC-seq PanCan dataset

| datatype | ATACeq |
|---|---|
| dataMatrix | TCGA_ATACseq.mat |
| dataFormat | mat |
| geneIDType | |
| logTransformed | yes |
| rangeK | 2:30 |
| repTimes | 10000 |

TABLE 5
Configure file for single-sample weight
estimation of the COMPASS dataset

| | |
|---|---|
| refSet | TCGA_RNAseq_PAAD |
| dataMatrix | COMPASS_PDAC.tsv |
| dataFormat | tsv |
| geneIDType | geneSymbol |
| logTransformed | no |

TABLE 6
Configure file for single-sample weight
estimation of the ICGC dataset

| | |
|---|---|
| refSet | TCGA_RNAseq_PAAD |
| dataMatrix | ICGC_PancreaticCancer.tsv |
| dataFormat | tsv |
| geneIDType | geneSymbol |
| logTransformed | no |

### Example 1

### PDAC Compartments Faithfully Captured in Microarray Data Set

Given a bulk measurement A, which is a N×M matrix of N rows of genes and M columns of samples, the aim of the de novo deconvolution is to factorize A into two matrices W and H, where W is a N×K matrix, H is a K x M matrix, and K is the number of compartments (FIG. 1B). Each compartment is associated with an overrepresented biological process or cell type, with W recording the gene weights measuring the relevance of each gene for each compartment and H recording the compartment weights measuring the relevance of each compartment for each sample. To derive stable W and H for optimal compartments, and circumvent the need to heuristically determine K, DECODER was developed as a sophisticated framework which integrates multiple runs of a carefully designed NMF-based pipeline based on an increasing number of factors (\tilde{K}). In each run, a gene weight seed (W′) is first trained by R iterations of the NMF algorithm, followed by applying final NMF and nonnegative least squares (NNLS) projection (FIG. 1C, see the Methods section). With the deconvolved factors at multiple runs of increasing \tilde{K}, factor linkages are established by linking the most associated factors between adjacent runs. Finally, compartments are determined dynamically by evaluating factor scores and score patterns along each branch of linked factors, allowing compartments to be located at different runs of \tilde{K} (FIG. 1D, see the Methods section). For this study, factors are used to refer to the direct output from each run at \tilde{K}, while compartments are used to refer to the final identified biological components from all the factors.

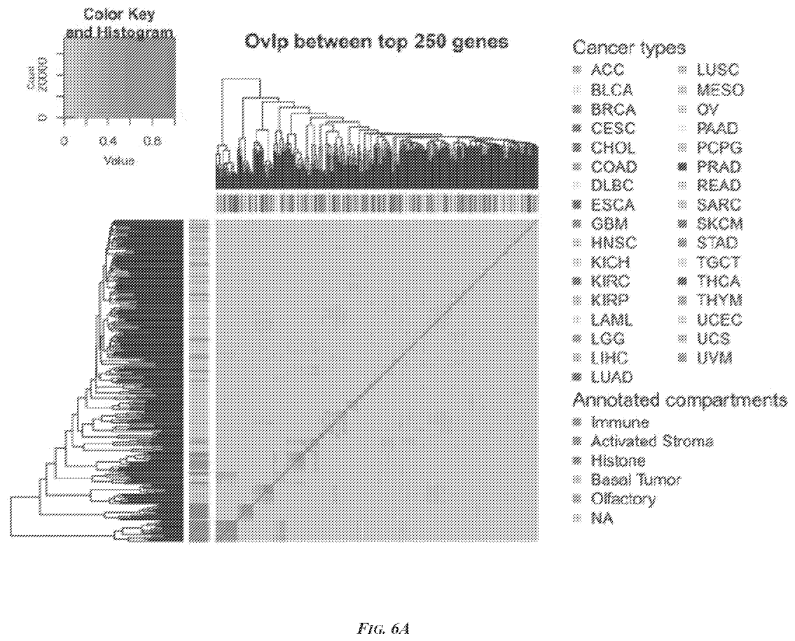

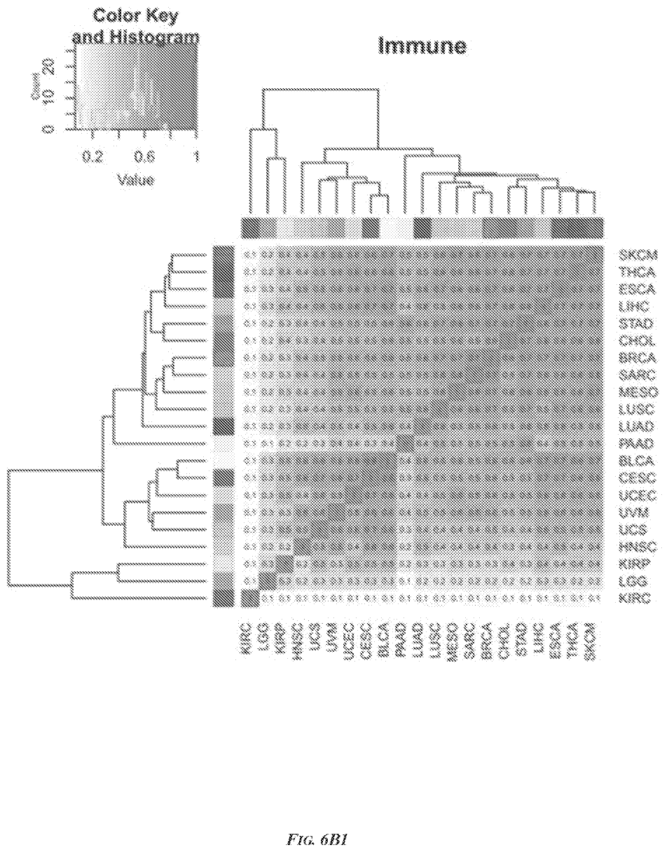

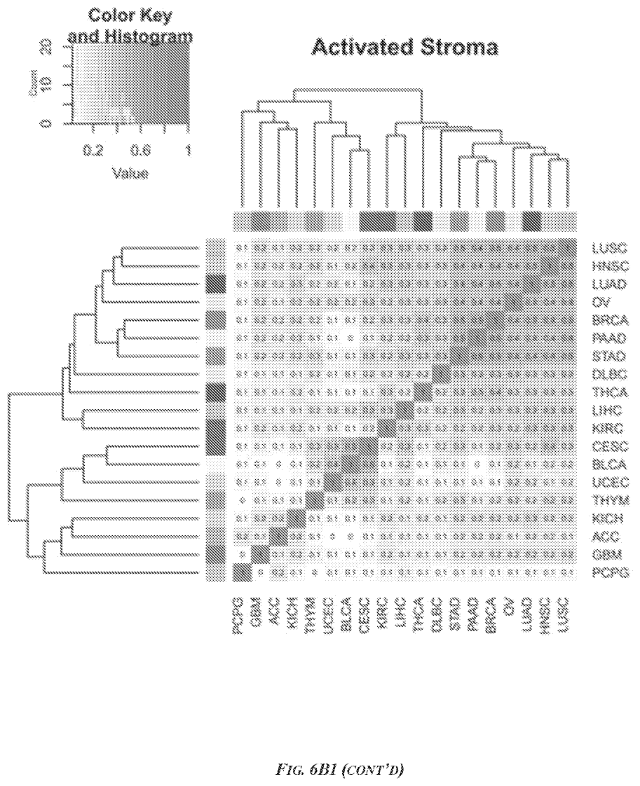

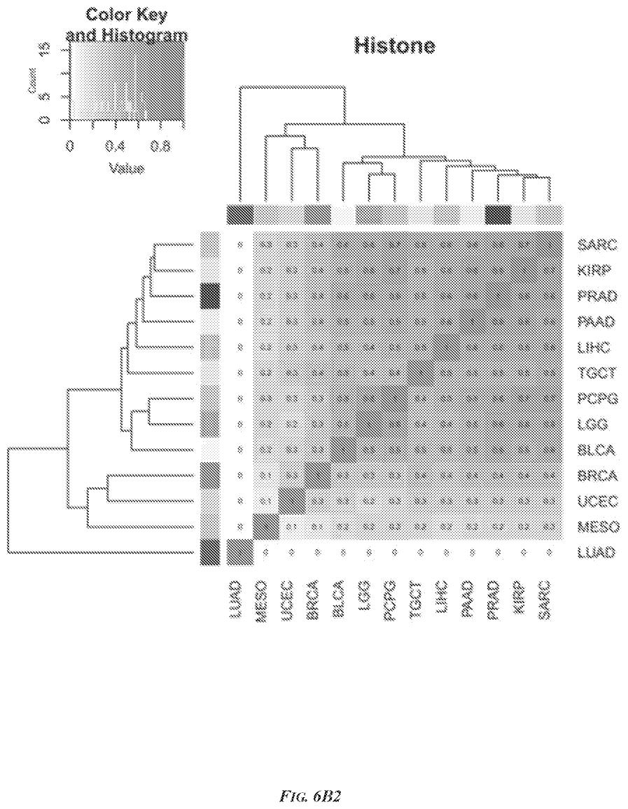

In a PDAC microarray data set containing primary tumor, metastatic and normal samples, DECODER identified 14 major compartments as a blinded determined solution, which accurately reproduced each of the compartments deconvolved previously using NMF at K=14 based on empirical trialing (Moffitt et al., 2015; FIG. 1E). For the matched compartments in the current and previous result, the gene weights show good correlation (median: 0.847, range: 0.761-0.924), and the top 250 genes show a large overlap (median: 158, range: 85-207; FIGS. 5A and 5B). Similar to previous results, enrichment of deconvolved compartment weights for patient samples exhibit excellent agreement with known tissue labels (Moffitt et al., 2015; FIG. 5C). Notably, metastatic samples show enriched weights for compartments of both the metastatic and primary sites, e.g., liver metastases show enriched DECODER weights for the liver compartment, as well as PDAC basal tumor and classical tumor compartments. This is in agreement with other studies, where liver metastases were found to be molecularly conserved compared with their primary tumors in PDAC (Connor et al., 2019; Martens et al., 2019). The high level of agreement is reassuring and suggests that DECODER may be used to enable the automated identification of compartments instead of involving labor-intensive manual annotation and empirical determination of K at multiple separate runs.

### Example 2

### Deconvolution of 33 TCGA Cancer Types

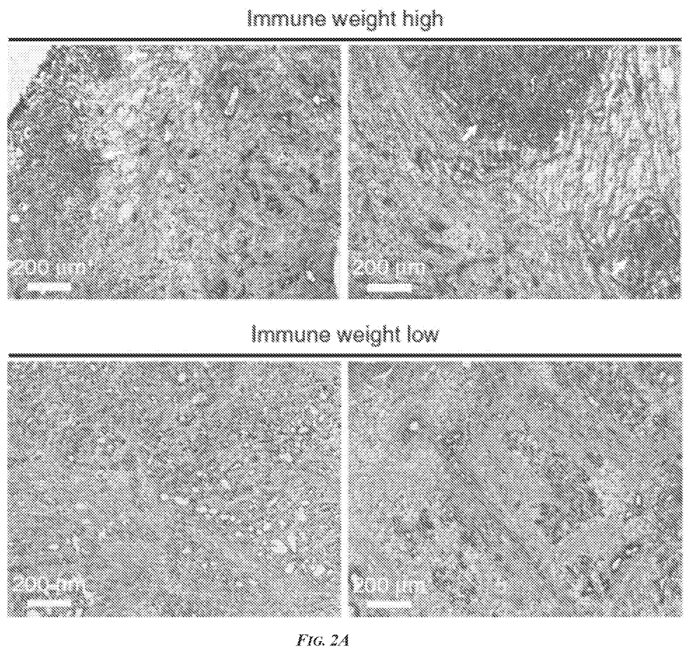

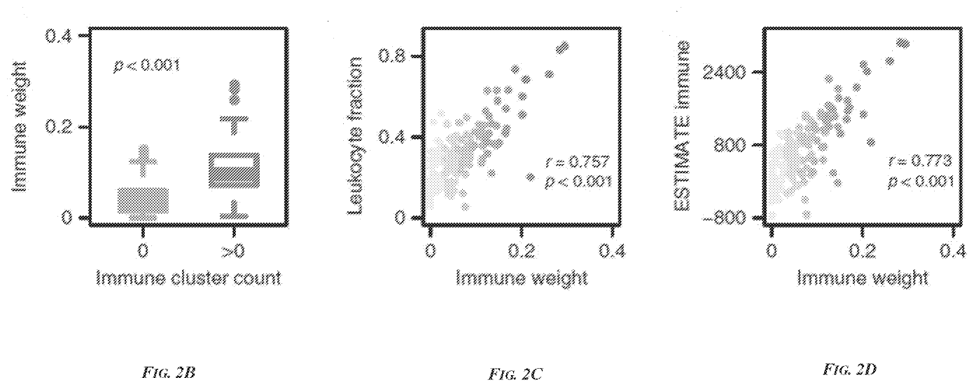

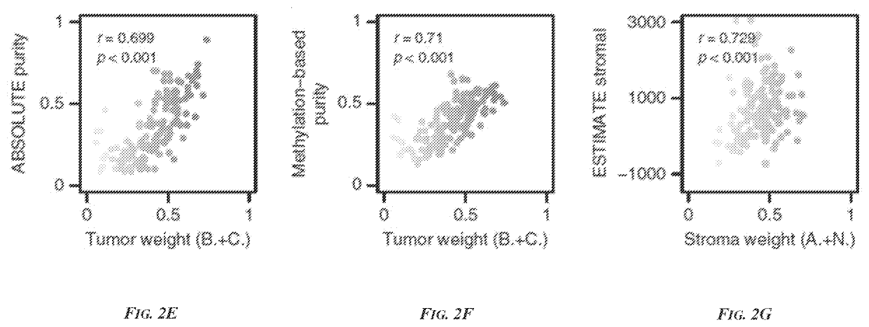

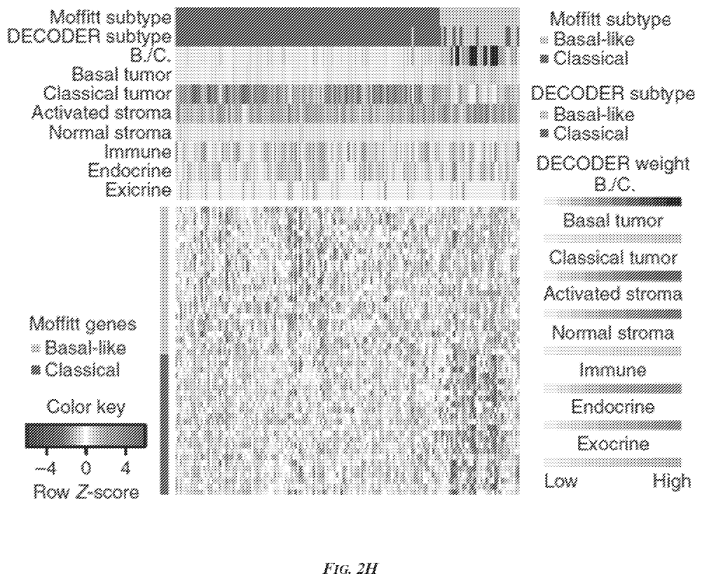

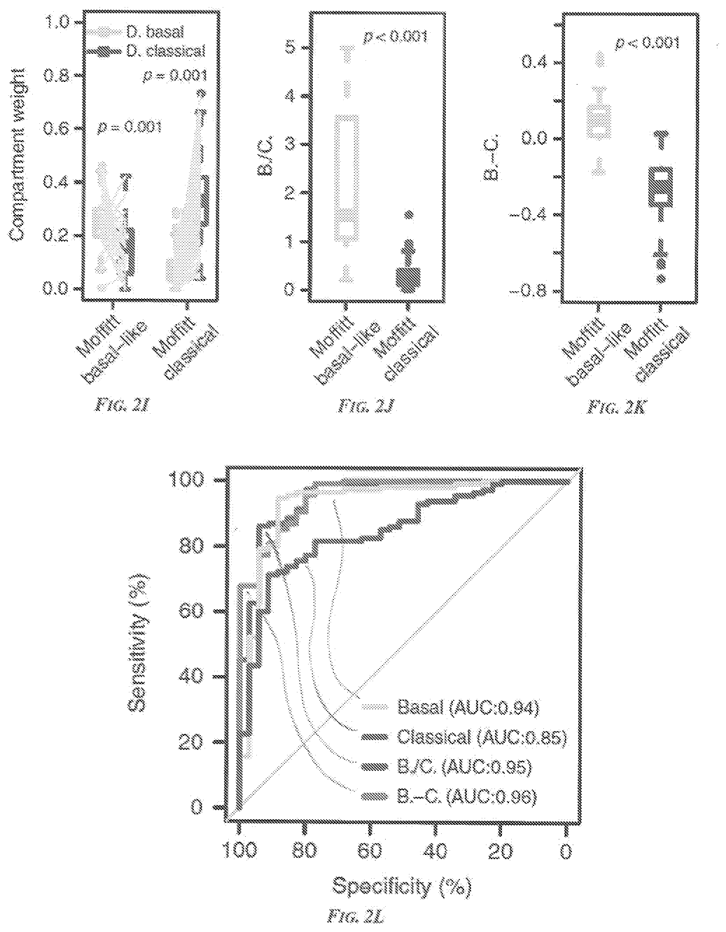

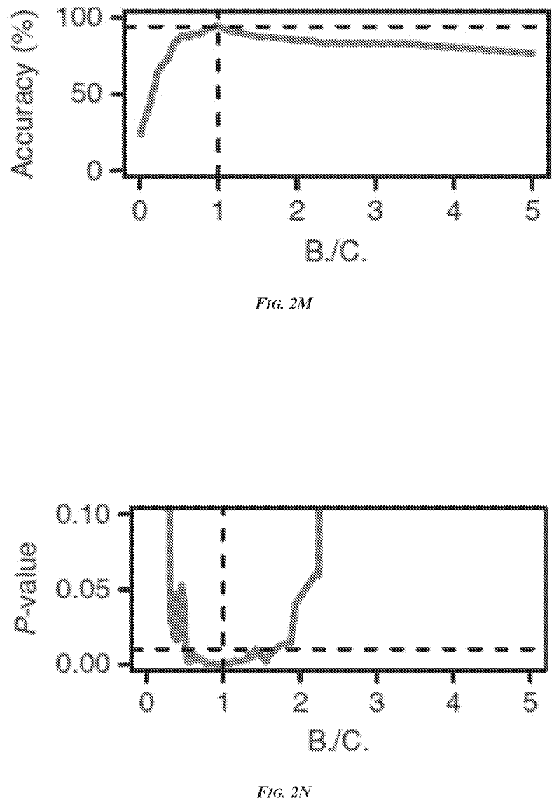

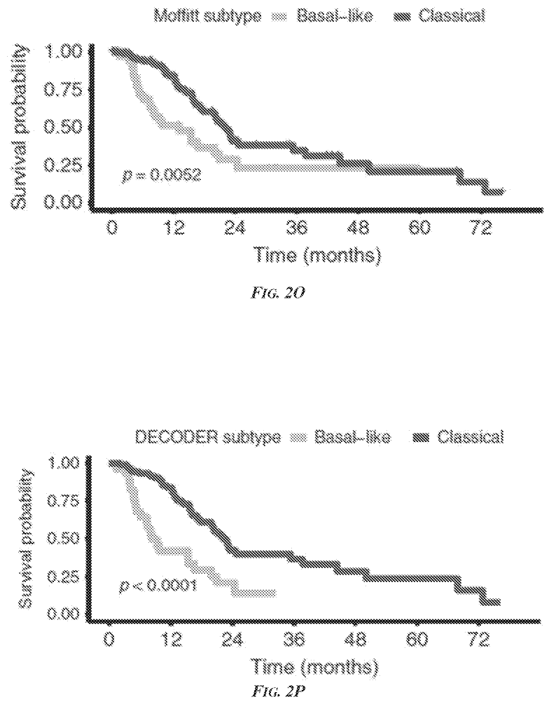

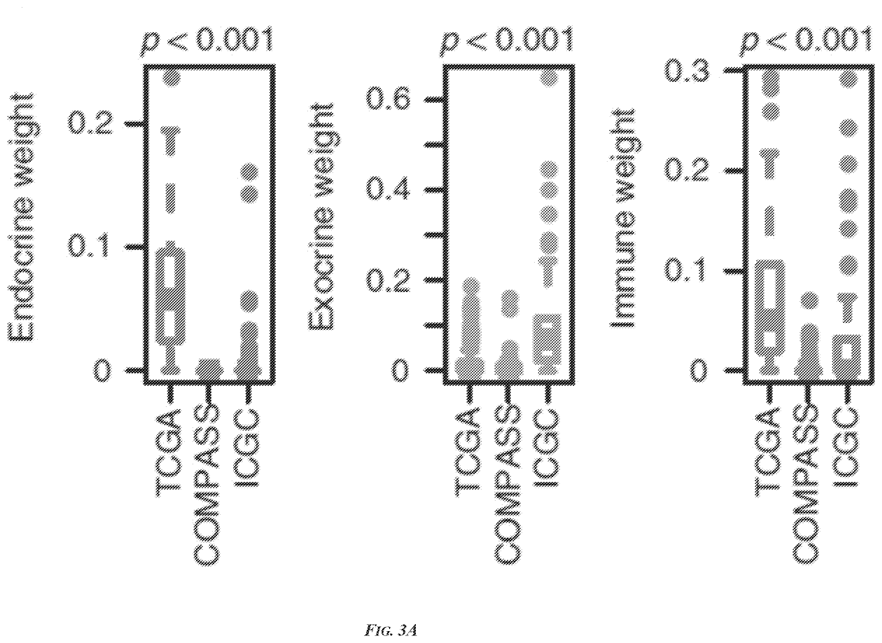

We then applied DECODER to each of the TCGA RNA-seq data sets of 33 cancer types for de novo compartment deconvolution. The compartments identified within cancer type were pooled, resulting in 269 compartments in total, with a median of 9 compartments in each cancer type (range: 4-14; Complete results available on GitHub: https://github.com/laurapeng/decoder/results). Then the ranked list of genes for each compartment was subjected to gene set analysis using the Molecular Signatures Database v3.1 (MSigDB; Subramanian et al., 2005) for the biological interpretation and annotation of compartments. We manually annotated five compartments common across cancer types by using MSigDB: immune, basal tumor, activated stroma, histone, and olfactory. Analysis of overlap in the top 250 genes of 269 compartments showed four clusters of closely correlated compartments across cancer types: immune, activated stroma, histone, and basal tumor (FIGS. 2A and 2B).

For the TCGA pancreatic adenocarcinoma (PAAD) data set, nine major compartments were identified using DECODER de novo deconvolution. We analyzed each compartment by examining enriched MSigDB gene sets, and found that seven of the nine were dominant compartments similarly defined in the microarray data set for PDAC: basal tumor, classical tumor, activated stroma, normal stroma, immune, endocrine, and exocrine (FIG. 1F). For each compartment, genes with distinctly large weights were selected as marker genes (FIG. 7A, Data Table 2, see the Methods section). To investigate the representativeness of the marker genes, we used a linear model described by DSA (Zhong et al., 2013) to calculate the fractions of seven dominant compartments using these marker genes. We found that the leukocyte fraction and ESTIMATE immune score were correlated with the estimated immune compartment fraction (FIGS. 7B and 7C). In addition, both of the ABSOLUTE and methylation-based tumor purity were highly correlated with the sum of the basal tumor and classical tumor compartment fractions (FIGS. 7D and 7E), and the ESTIMATE stromal score was highly correlated with the sum of the activated stroma and normal stroma fractions (FIG. 7F). These findings demonstrate that DECODER can automatically and robustly determine biological compartments in a given data set de novo, and identify representative marker genes for each compartment.

### Example 3

### Compartment Weights (H) Derived From De Novo Deconvolution

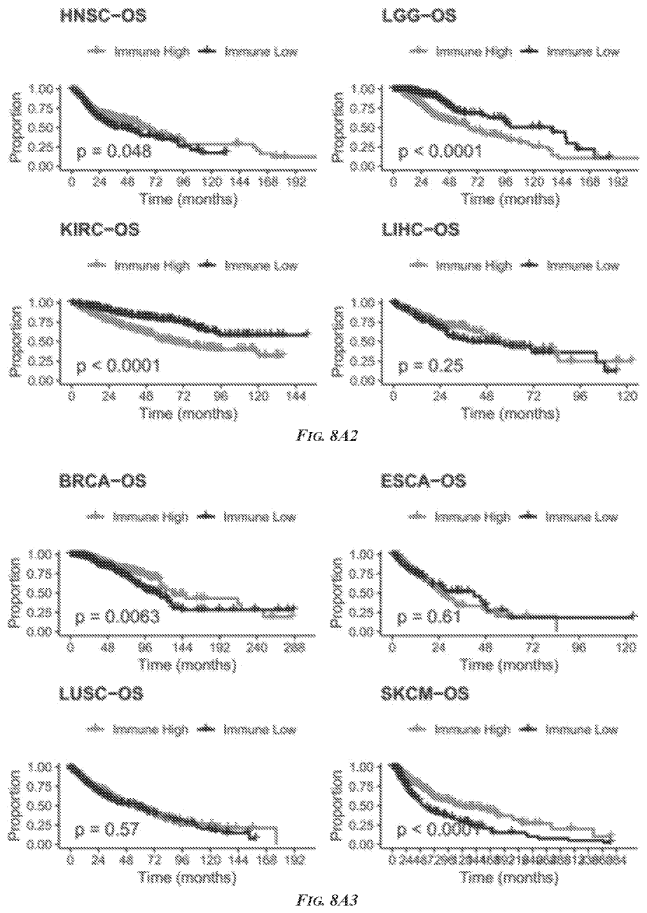

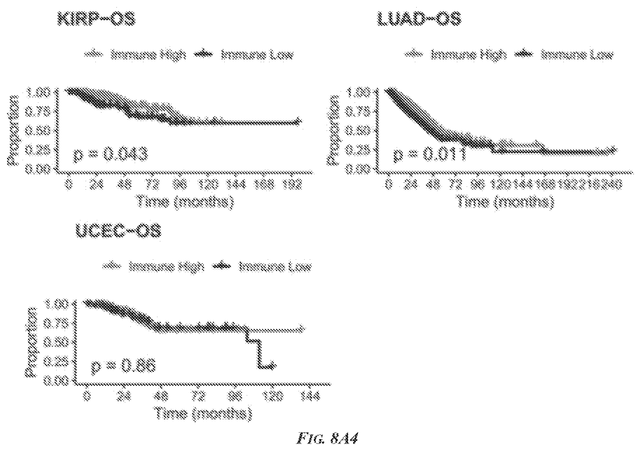

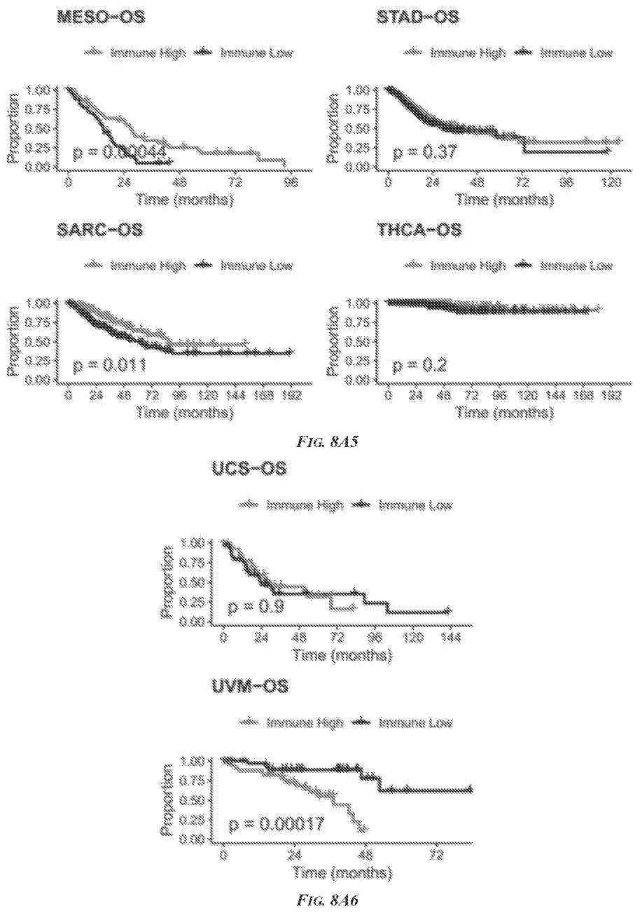

A matrix of compartment weights for samples (H) was also derived from the de novo deconvolution. We hypothesized that for each sample, a larger weight is associated with a larger representation for a specific compartment. Indeed, in TCGA PAAD, when we evaluated the hematoxylin and eosin (H&E) slides for the presence or absence of immune infiltrate and tertiary lymphoid structures within the tumor, samples with high immune weights showed apparent infiltration, which was absent in low immune-weighted samples (FIG. 2A). Quantitatively, samples containing tertiary lymphoid structures (n=37) showed significantly higher immune weights than those with no tertiary lymphoid structures (n=84; FIG. 2B). We also found that normalized DECODER weight for the immune compartment was highly correlated with leukocyte fraction (FIG. 2C) and ESTIMATE immune score (FIG. 2D). In addition, high immune weights predict better overall survival in TCGA PAAD, as well as a subset of the 33 cancer types (Liu et al., 2018; FIGS. 8A1-8A6). For tumor compartments, we demonstrated high correlation between the sum of basal tumor and classical tumor weights, and tumor purity based on both ABSOLUTE (FIG. 2E) and methylation (FIG. 2F). Similarly, the ESTIMATE stromal score is mirrored by the sum of activated stroma and normal stroma weights (FIG. 2G).

Based on previous tumor subtype calls (the Moffitt schema) derived by consensus clustering using exemplar genes for these samples (The Cancer Genome Atlas Research Network, 2017), we show that Moffitt Basal-like samples (n=37) are associated with higher DECODER basal compartment weights and Moffitt Classical samples (n=113) with higher DECODER classical compartment weights (FIGS. 2H and 2I). Hereafter, for clarity, basal and classical tumor (lowercase) refer to DECODER-derived compartments, and Basal-like and Classical (uppercase) refer to the categorical subtypes. Similarly, higher ratios or differences of basal versus classical tumor compartment weights are associated with Moffitt Basal-like subtypes (FIGS. 2J and 2K). To determine the clinical significance of basal versus classical tumor compartment weights, we compared the utility of compartment weights as a clinical variable. We found that the ratio (B./C.; p=0.049, Cox proportional-hazards model) but not difference (B.-C.; p=0.073, Cox proportional-hazards model) between DECODER basal and classical tumor weight is associated with survival as continuous variables.

To determine the accuracy of using DECODER compartment weights to perform binary classification of tumor subtypes, we calculated the area under the receiver operating curve (AUC) using Moffitt tumor subtype calls as the gold standard. We found that basal compartment weight alone, B./C., and B.-C., have similarly high area under the receiver-operating curve (AUC 0.94-0.96; FIG. 2L). Because B./C. shows the second highest AUC after B.-C. and is associated with outcome as a continuous variable, we then determined a threshold for the ratio (B./C.=1) to reach optimal accuracy to call Moffitt subtypes (FIG. 2M), and optimal significance to differentiate patient outcome (FIG. 2N). Interestingly, the subtype-specific survival differences between the Basal-like (n=28) and Classical samples (n=122) were even more pronounced for the calls derived by DECODER B./C. than by the consensus clustering based calls of Moffitt schema (FIGS. 2O and 2P).

### Example 4

### Single-Sample Compartment Weight Estimation

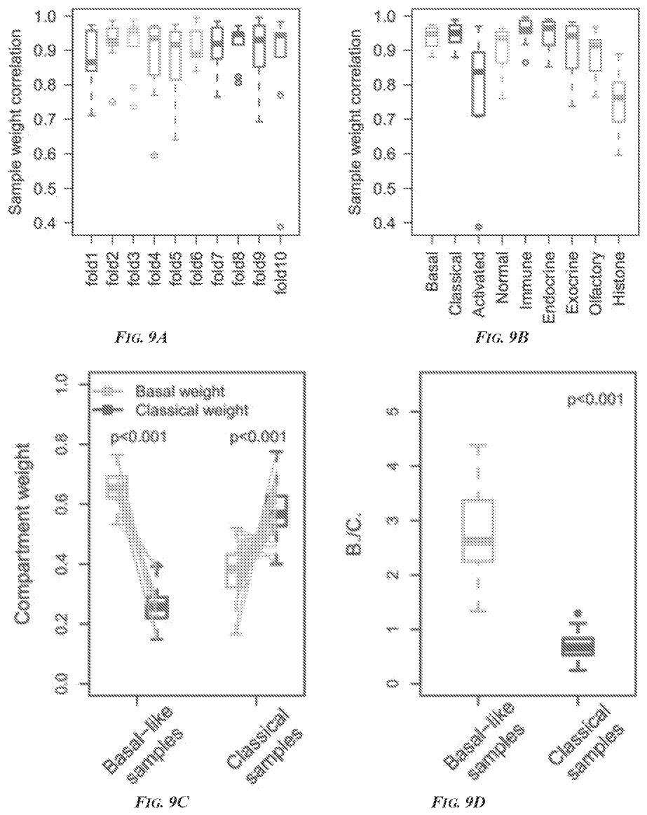

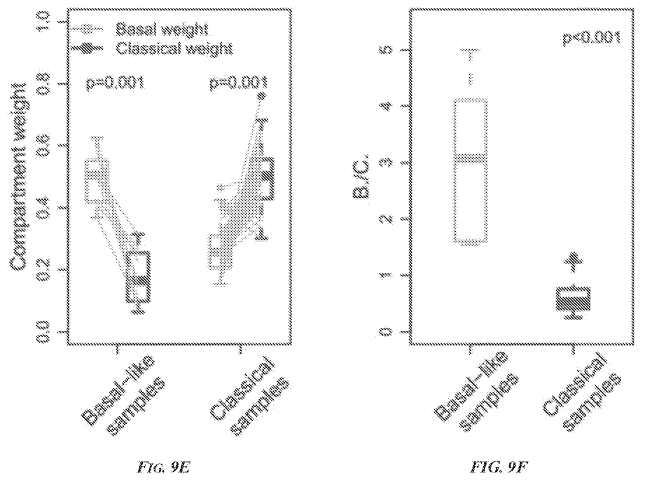

Since the compartment weights for TCGA data set were obtained through de novo deconvolution which requires a data set with multiple samples and relatively large amounts of computing time during the training process, we next developed a method to deconvolve RNA-seq samples and calculate the compartment weights without the need to apply the de novo deconvolution. This method was built using the deconvolved gene weights for compartments (W) and applying NNLS to indicate the compartment weights for even a single sample (see the Methods section). Ten-fold cross-validation demonstrated good reproducibility of compartment weights derived from gene weights compared with those derived from de novo deconvolution (FIGS. 9A and 9B). We applied this algorithm to calculate the compartment weights for the COMPASS trial (Aung et al., 2018) and ICGC PACA-AU RNA-seq data sets (Bailey et al., 2016) based on gene weights derived in de novo deconvolution of the TCGA PAAD data set .

Interestingly, in COMPASS (n=50), where samples were microdissected, the endocrine, exocrine, and immune weights were significantly lower than those in the TCGA PAAD and ICGC data sets (FIG. 3A). In the ICGC data set (n=70), similar to our TCGA PAAD results, we found that ABSOLUTE-based tumor purity, ESTIMATE-based immune and stromal scores correlate with respective compartment weights derived by DECODER (FIGS. 3B-3D). For the two acinar cell carcinoma samples in this data set, we found that the exocrine weights were 4.78-fold and 6.96-fold higher than the mean of the exocrine weights in all other samples. This suggests that the calculated DECODER weights can accurately capture the biological composition of a sample. In addition, the ICGC-subtyped ADEX samples (n=9) are associated with higher exocrine weights, and the immunogenic samples (n=20) are associated with higher immune weights (FIG. 3E). These results may suggest that ADEX and Immunogenic samples are characterized by an overrepresentation of exocrine and immune cells in the respective samples, which may confound the identification of tumor-specific subtypes (The Cancer Genome Atlas Research Network, 2017; Puleo et al., 2018).

In both the COMPASS and ICGC data sets, the DECODER basal weight, classical weight, as well as B./C. were associated with the Moffitt subtypes (FIGS. 3F and 3G and FIGS. 9C-9F). As expected, invasive intraductal papillary mucinous neoplasm (IPMN, n=10) showed the lowest B./C., while the adenosquamous carcinoma samples (n=4) showed the highest, suggesting that the B./C. is able to identify the extremes of tumor histology (Collisson et al., 2019; FIG. 3H). In the ICGC data set, where survival data were available, the DECODER Basal-like (n=18) and Classical samples (n=52) subtyped by B./C. were differentially associated with patient outcome (FIG. 3I). In addition, B./C. was found to significantly correlate the percentage of tumor size change after treatment in DECODER Basal-like subtype samples in COMPASS, where patients were treated with either modified-FOLFIRINOX (FFX) or gemcitabine plus nab-paclitaxel (GP; FIG. 3J). However, this trend was not observed in the DECODER Classical subtype samples (FIG. 3K). These results demonstrated that the DECODER compartment weights may faithfully and accurately recapitulate the differences in the biological make-up of a single sample, thus enabling the accurate prediction of clinical variables.

### Example 5

### Application on TCGA PanCan ATAC-Seq Data Set

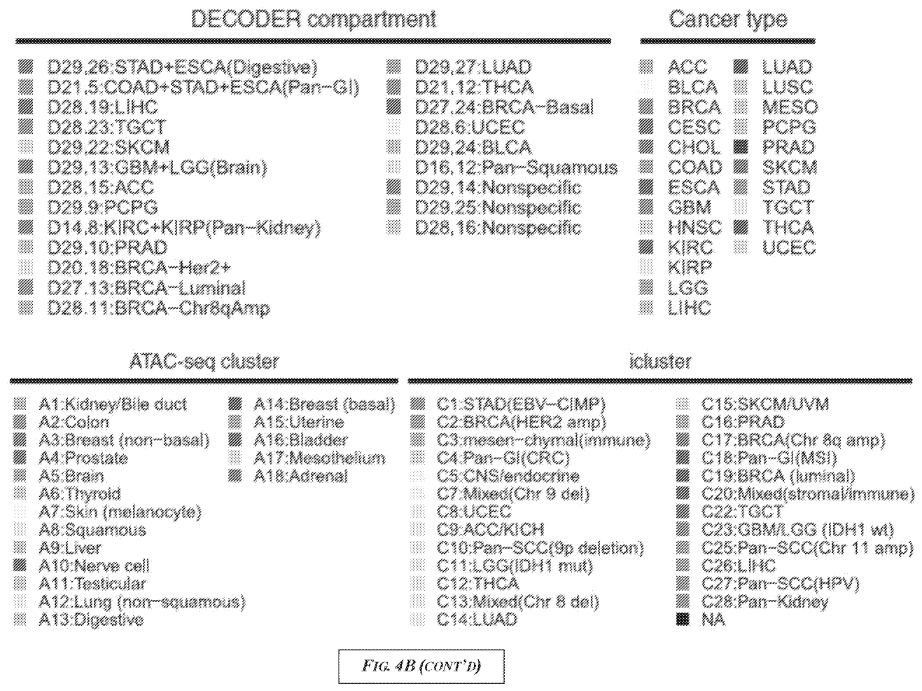

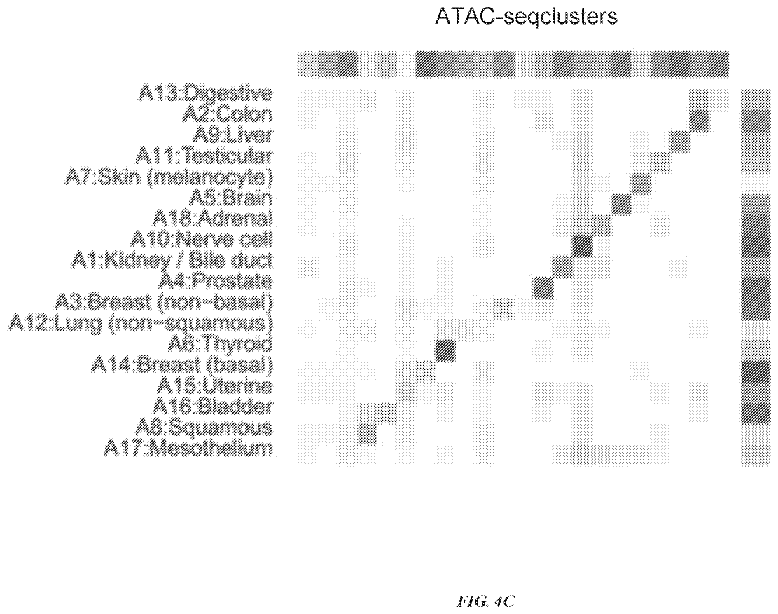

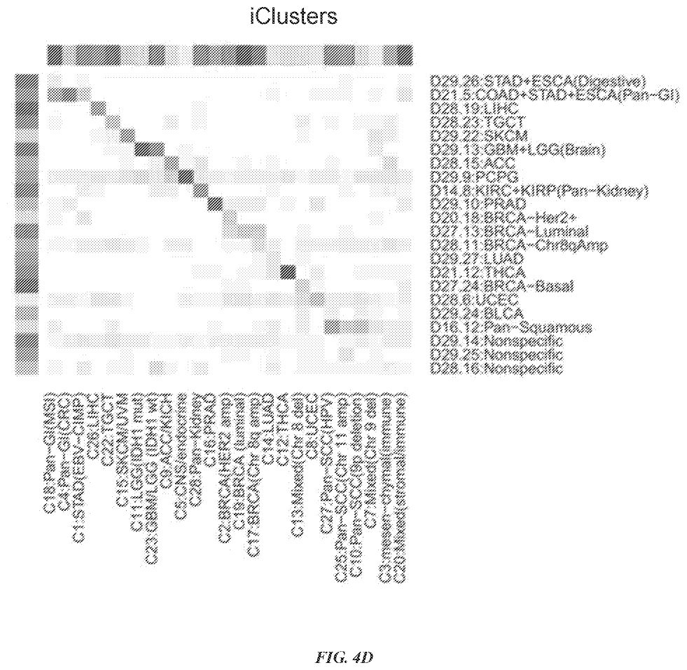

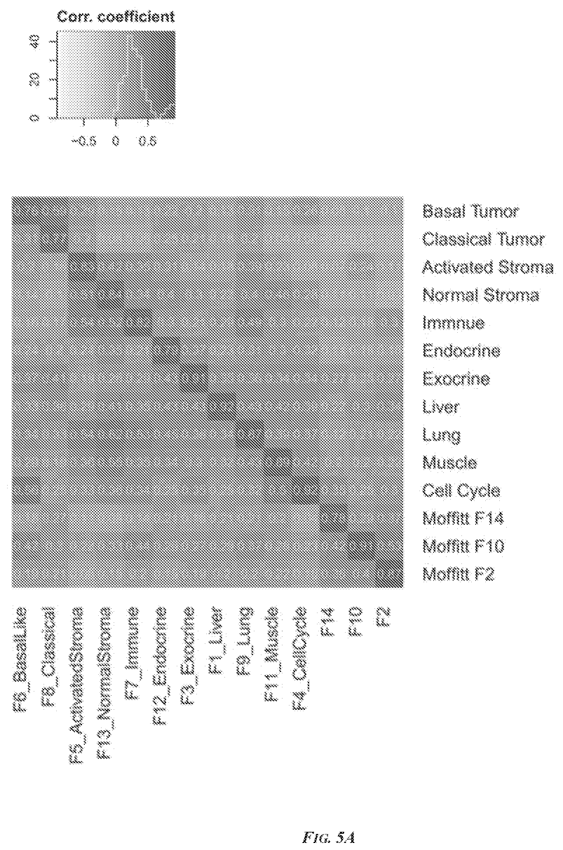

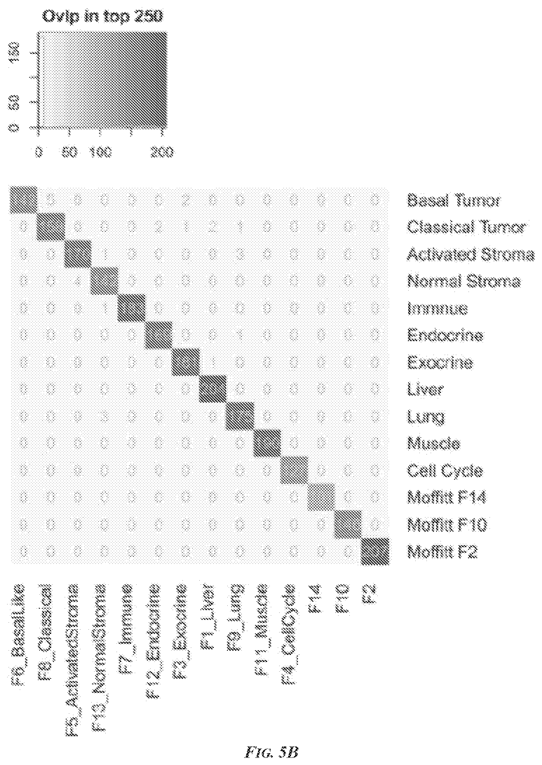

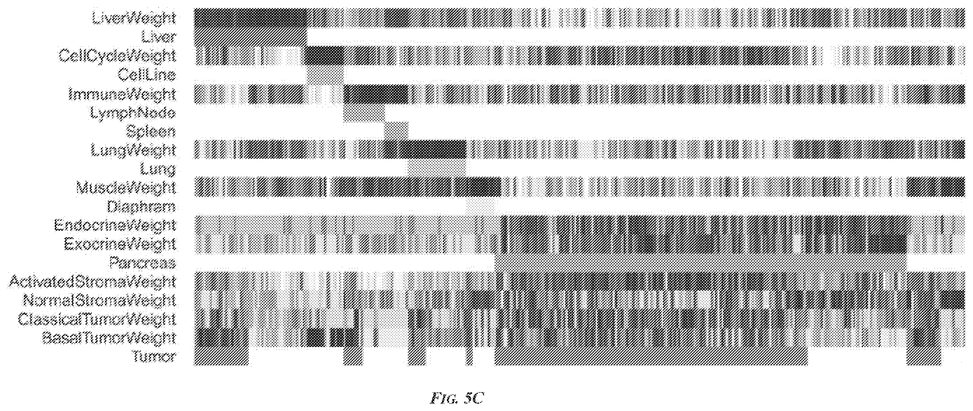

Previous PanCan analysis on an ATAC-seq data set of 23 human cancers has identified 18 distinct clusters of samples, which showed strong concordance with the published multiomic iCluster scheme (Corces et al., 2018; Hoadley et al., 2018). These studies found both homogeneous clusters for single-tumor types, and heterogeneous clusters formed by mixed-tumor types arising from the same organ systems or with similar features, with some cancer types split into multiple clusters. We therefore interrogated whether DECODER can deconvolve the PanCan ATAC-seq data set containing 759 replicates from 410 unique samples into compartments that reflect inner biological composition. DECODER identified 22 major compartments. Unsupervised clustering on normalized compartment weights revealed clusters of known labels of cancer types, ATAC-seqbased clusters and iClusters (FIG. 4A). For example, the compartment denoted as D28.19:LIHC was found to show enriched weights in liver hepatocellular carcinoma (LIHC) samples (FIG. 4A), as well as in the ATAC-seq based cluster A9:Liver and the iCluster C26:LIHC (FIGS. 4A, 4C, 4D).

Ten compartments were found to show uniquely higher weights in single cancer types, namely LIHC (D28.19:LIHC), skin cutaneous melanoma (D29.22:SKCM), adrenocortical carcinoma (D28.15:ACC), pheochromocytoma and paraganglioma (D29.9:PCPG), prostate adenocarcinoma (D29.10:PRAD), thyroid carcinoma (D21.12:THCA), uterine corpus endometrial carcinoma (D28.6:UCEC), testicular germ cell tumors (D28.23: TGCT), lung adenocarcinoma (D29.27:LUAD), and bladder urothelial carcinoma (D29.24:BLCA)(FIG. 4B). Similarly, we found their compartment weights to be the highest for ten respective ATAC-seq clusters and iClusters (FIGS. 4C and 4D). This suggests that DECODER identified cancer-specific compartments similar to previous findings which identified cancer-specific clusters using ATAC-seq-based clustering and iClusters (Corces et al., 2018; Hoadley et al., 2018).

DECODER also identified organ system-associated compartments. For instance, brain (D29.13:GBM+LGG[Brain]), pan-kidney (D14.8:KIRC+KIRP[Pan-Kidney]), pan-gastrointestinal (D21.5:COAD+STAD+ESCA[Pan-GI]), digestive (D29.26:STAD+ESCA[Digestive]), and pan-squamous (D16.12:Pan-Squamous). For both of the brain tumors (glioblastoma multiforme [GBM] and brain lower grade glioma [LGG]), D29.13:GBM+LGG(Brain) showed exclusively high weight. Intriguingly, the compartment of D29.9:PCPG also showed relatively high weight in GBM and LGG, reflecting the fact that they may be anatomically similar. Comparing with ATAC-seq clusters and iClusters, D29.13:GBM+LGG(Brain) was associated with A5:Brain, and C11:LGG(IDH1 mut) or C23:GBM/LGG(IDH1 wt), respectively. D14.8:KIRC+KIRP(Pan-Kidney) was distinctly highly weighted for kidney clear cell carcinoma (KIRC) and kidney renal papillary cell carcinoma (KIRP), represented as a pan-kidney compartment, which is associated with A1:Kidney/Bile duct and C28:Pan-Kidney. Similarly, D21.5:COAD+STAD+ESCA(Pan-GI) and D29.26:STAD+ESCA(Digestive) were found to be related to the pan-GI system and clusters, with D21.5:COAD+STAD+ESCA(Pan-GI) exhibiting the highest weights in colon adenocarcinoma (COAD) and stomach adenocarcinoma (STAD), and the second highest weights in esophageal carcinoma (ESCA). For ESCA, which may have squamous morphology components, the compartment with highest weight was annotated as the pan-squamous compartment (D16.12:Pan-Squamous), showing the strongest associations with the squamous clusters in ATAC-seq and iCluster as well. D16.12:Pan-Squamous is also the most highly represented in head and neck squamous cell carcinoma (HNSC), lung squamous cell carcinoma (LUSC), and cervical and endocervical cancers (CESC), known to have squamous histologies. Interestingly, while D16.12:Pan-Squamous showed the second highest weights in BLCA, we found that the cancer-specific D29.24:BLCA compartment was overrepresented as well. This is in agreement with the finding that BLCA has very diverse iCluster memberships (Hoadley et al., 2018).

Compartments D27.24:BRCA-Basal, D20.18:BRCA-Her2+, D28.11:BRCA-Chr8qAmp, and D27.13:BRCA-Luminal were all found to be associated with breast invasive carcinoma (BRCA) (N=141), enabled by sample sufficiency in the data set and as expected by its known heterogeneity (The Cancer Genome Atlas Network, 2012). Less prominent compartments were found in cholangiocarcinoma (CHOL, n=10), TGCT (n=18), and mesothelioma (MESO, n=13), which may be due to the small samples sizes available.

### Example 6

DECODER has identified 7 reproducible compartments in pancreatic ductal adenocarcinoma (PDAC), namely basal-like tumor, classical tumor, activated stroma, normal stroma, immune, endocrine and exocrine using RNA-seq gene expression data. DECODER has also identified compartment-specific genes for each of these compartments. The gene lists can be used for the calculation of relative compartment fractions in bulk tumor samples, using published linear models, e.g. DSA³⁶. In addition, all the genes in each compartment are sorted by their importance, so that the higher the rank of the gene in a certain compartment, the more specific the gene is to the compartment. Therefore, the top ranked genes can be used as staining markers by pathologists to identify and distinguish the 7 compartments in PDAC.

A table of gene weights for each compartment in PDAC was derived as the PDAC RNA-seq reference matrix by apply de novo DECODER to TCGA PAAD dataset. Using this reference matrix, compartment weights for a PDAC RNA-seq sample can be estimated using the single-sample deconvolution utility of DECODER. The compartment weights closely reflect relative fractions of compartments in a sample. This process is single-sample based, which is more desirable than the linear-model based approaches in the clinical setting. Importantly, each compartment weight of DECODER is associated with strong biological and clinical implications. The higher immune weight is highly indicative of presence of immune infiltrate and tertiary lymphoid structures. The exocrine and endocrine weights are indicative of normal tissue contamination in the tumor sample. The sum of activated and normal stroma weights represents the amount of the stroma, and the sum of basal-like and classical tumor weights represents the purity of the tumor sample. In addition, the ratio between the basal-like and classical tumor compartments (bcRatio) were proved to be associated with patient outcome and treatment response. If bcRatio is used to call binary subtypes, the basal-like subtype with bcRatio >1 will show significantly worse prognosis. As a continuous variable, higher bcRatio can predict shorter survival time. In addition, this bcRatio is highly predictive of tumor volume change upon chemotherapy in basal-like patients.

DECODER can also be used to identify the tumor of origin for cancers of unknown primary. A table of ATAC-seq genomic region based weights was derived as the PanCan ATAC-seq reference matrix by applying de novo DECODER to PanCan ATAC-seq dataset. In this matrix, compartments are defined as multiple cancer types or organ systems. With the tag intensity in the open chromatin regions of a tumor sample measured by ATAC-seq, DECODER can use the reference matrix to estimate the compartment/cancer weights of the sample. The tumor of origin will be identified to be the compartment/cancer showing the highest weights.

### Discussion of the EXAMPLES

Tumors are mixtures of different compartments. While global gene expression analysis profiles the average expression of all compartments in a sample, identifying the specific contribution of each compartment remains a challenge. With the increasing recognition of the importance of non-neoplastic components, the ability to breakdown the gene expression contribution of each is critical. Here, we develop DECODER, an integrated framework which performs de novo deconvolution and single-sample compartment weight estimation. We use DECODER to deconvolve 33 TCGA tumor RNA-seq data sets and show that it may be applied to other data types including ATAC-seq. We demonstrate that it can be utilized to reproducibly estimate cellular compartment weights in pancreatic cancer that are clinically meaningful. Application of DECODER across cancer types advances the capability of identifying cellular compartments in an unknown sample and may have implications for identifying the tumor of origin for cancers of unknown primary.

DECODER is an integrated framework, which we developed to perform de novo compartment deconvolution for any data set with nonnegative values, and conduct efficient compartment weight estimation for even a single sample of cancer types in TCGA. Standard NMF methods pre-define the number of factors (K) and assume the presence of K compartments in a data set. In contrast, the de novo compartment deconvolution of DECODER is fluid and allows each of the compartments in a data set to be identified at runs of different \tilde{K}, facilitating the identification of each compartment to be more robust. This obviates the need for prior knowledge of the compartments and the number of them in a data set, since compartments vary in different cancers or tissues and certain compartments present in one may be absent in another.

We applied DECODER to deconvolve each of the 33 cancer types in the TCGA RNA-seq data sets. This has resulted in the identification of cancer-type-specific marker genes, which may lead to more accurate estimation of certain compartment fractions. As far as we know, current methods for deconvolution of tumor, stroma, and immune fractions use the same set of curated marker genes for all cancer types (Carter et al., 2012; Newman et al., 2015). Furthermore, cancer-specific marker genes may be studied as cancer-specific biomarkers. The deconvolved mRNA compartments and respective marker genes for 33 cancer types are readily accessible on GitHub [https://github.com/laurapeng/decoder/results].

Our detailed examination on the resultant compartments of TCGA PAAD data set demonstrated the robust identification of biological compartments in PDAC, as defined by previously known knowledge (Moffitt et al., 2015). We also showed that DECODER compartment weights for tumor, stroma, and immune were highly correlated with respective measurements by previous independent methods based on copy-number variations, methylation, and expression. In addition to the previously described compartments of basal-like tumor, classical tumor, activated stroma, normal stroma, immune, endocrine, and exocrine, we also identified two new compartments, which we annotated as olfactory and histone. Interestingly, a study has associated the olfactory transduction pathway with pancreatic cancer risk (Wei et al., 2012), and additional studies have found overexpression of olfactory receptors in multiple cancer types, including prostate (Wang et al., 2006; Weng et al., 2006), bladder (Weber et al., 2018a), and breast (Weber et al., 2018b) cancers. In agreement with these studies, we found that our olfactory compartment was indeed present in prostate adenocarcinoma (PRAD), bladder urothelial carcinoma (BLCA), as well as several other cancer types . Similarly, the histone compartment was identified in multiple cancer types. Further investigation will be required to determine if the olfactory and histone compartments are true biological compartments or artifacts of inherent noise in RNA-seq.

Unlike regular clustering-based methods, DECODER provides the possibility of examining a sample multidimensionally via the compartment weights, instead of forcing a given sample to a specific cluster. This provides more detailed information regarding the biological composition of a sample, which can be used for comparison across samples and data sets. In PDAC, absolute consensus for transcriptomic subtypes by different taxonomies has not been achieved, with the exocrine-like/ADEX and immunogenic subtypes at the center of the controversies (Collisson et al., 2019). By applying DECODER to the COMPASS and ICGC data sets, we demonstrated that in COMPASS, where samples were microdissected, there is comparatively less of the exocrine and immune compartments. In ICGC, the exocrine compartment weights correlate with the exocrine-like/ADEX subtype while immune compartment weights correlate with the Immunogenic subtype. These findings agree with the association of low-purity samples with exocrine-like/ADEX and Immunogenic subtypes (The Cancer Genome Atlas Research Network, 2017) and suggest that the exocrine-like/ADEX and immunogenic subtypes may be explained by the presence of the non-tumor compartments. In addition, in the more heterogenous ICGC data set, basal and classical tumor weights correlate well with IPMN versus adenoquamous carcinomas. Therefore, DECODER may facilitate the better elucidation of the underlying biology of molecular subtypes.

Furthermore, relying on the results from the initial de novo deconvolution in TCGA

PAAD, compartment weight estimation in ICGC and COMPASS was single-sample based, and therefore feasible in the clinical setting. Similarly, for any of the 33 cancer types in TCGA, DECODER can be used to estimate the compartment weights for a new given sample without the need to perform de novo deconvolution. Thus, DECODER is a powerful tool to break down a new tumor sample with known origin.

DECODER may be applied to any solid or hematologic malignancy, as well as liquid biopsies, and may also be used for platforms other than gene expression. From a computational perspective, DECODER could be applied to any data type with nonnegative values derived from multiple platforms, e.g. ChIPseq data, copy-number variations, DNA methylation data and single-cell sequencing platforms. As a proof of concept, we applied DECODER on the PanCan ATAC-seq data set containing 23 cancer types in a combined fashion, and identified compartments associated with cell-of-origin and organ systems, which highly reproduced previous clustering-based interpretations of the PanCan analysis (Corces et al., 2018; Hoadley et al., 2018). Similar to our application of DECODER in RNA-seq, deconvolution of the PanCan ATAC-seq data set can also be applied to a single new sample. An aspect of the PanCan analysis may be the unequal number of samples in different cancer types (range of number of samples: 7-141), which may lead to an unbalanced number of compartments identified for different cancers. Therefore, it is possible that more unbiased compartment information may be derived in the setting of a greater number of samples, especially for the cancers with smaller sample sizes.

From a clinical perspective, the ability of DECODER to identify sample compartments without the a priori assumption of an actual number of compartments, makes it a powerful tool for comparing tumor heterogeneity across samples and for the very challenging clinical conundrum of cancers of unknown primary. Cancers of unknown primary are metastatic cancers where the primary site of origin cannot be identified. Optimal treatment of these cancers remains a challenge, as knowledge of the primary site guides treatment decisions. We and others have previously shown that metastatic samples are molecularly conserved compared with their primary tumors (Moffitt et al., 2015; Connor et al., 2019). Our successful deconvolution on PanCan ATAC-seq data supports the fact that profiled open-chromatin regions are extremely tissue-specific (Sur & Taipale, 2016), making ATAC-seq a promising method for distinguishing a complex mixture of cancer types. More ATAC-seq cohorts in metastatic samples will be needed to further validate our results. The application of DECODER using ATAC-seq data may provide more biological information that can better guide treatment for cancers of unknown primary and will need to be studied in the context of future clinical trials.

### REFERENCES

All references cited in the instant disclosure, including but not limited to all patents, patent applications and publications thereof, scientific journal articles, and database entries (including but not limited to GENBANK® biosequence database entries and all annotations available therein) are incorporated herein by reference in their entireties to the extent that they supplement, explain, provide a background for, or teach methodology, techniques, and/or compositions employed herein.
1. Marusyk et al. (2012) Intra-tumour heterogeneity: a looking glass for cancer? Nat. Rev. Cancer 12:323-334.
2. Yadav & De (2015) An assessment of computational methods for estimating purity and clonality using genomic data derived from heterogeneous tumor tissue samples. Brief. Bioinform. 16:232-241.
3. Espina et al. (2007) Laser capture microdissection technology. Expert Rev. Mol. Diagn. 7:647-657.
4. Chung & Shen (2015) Laser capture microdissection: from its principle to applications in research on neurodegeneration. Neural Regen. Res. 10:897-898.
5. Wu et al. (2014) Quantitative assessment of single-cell RNA-sequencing methods. Nat. Methods 11:41-46.
6. Haque et al. (2017) A practical guide to single-cell RNA-sequencing for biomedical research and clinical applications. Genome Med. 9:75.
7. Carter et al. (2012) Absolute quantification of somatic DNA alterations in human cancer. Nat. Biotechnol. 30:413-421.
8. Zheng et al. (2014) MethylPurify: tumor purity deconvolution and differential methylation detection from single tumor DNA methylomes. Genome Biol. 15:419.
9. Zheng et al. (2017) Estimating and accounting for tumor purity in the analysis of DNA methylation data from cancer studies. Genome Biol. 18:17.
10. Ahn et al. (2013) DeMix: deconvolution for mixed cancer transcriptomes using raw measured data. Bioinformatics 29, 1865-1871.
11. Wang et al. (2015) UNDO: a bioconductor R package for unsupervised deconvolution of mixed gene expressions in tumor samples. Bioinformatics 31:137-139.
12. Yoshihara et al. (2013) Inferring tumour purity and stromal and immune cell admixture from expression data. Nat. Commun. 4:2612.
13. Gong & Szustakowski (2013) DeconRNASeq: a statistical framework for deconvolution of heterogeneous tissue samples based on mRNA-Seq data. Bioinformatics 29:1083-1085.
14. Newman et al. (2015) Robust enumeration of cell subsets from tissue expression profiles. Nat. Methods 12:453-457.
15. Zhong et al. (2013) Digital sorting of complex tissues for cell type-specific gene expression profiles. BMC Bioinforma. 14:89.
16. Moffitt et al. (2015) Virtual microdissection identifies distinct tumor- and stroma-specific subtypes of pancreatic ductal adenocarcinoma. Nat. Genet. 47:1168-1178.
17. Brunet et al. (2004) Metagenes and molecular pattern discovery using matrix factorization. Proc. Natl Acad. Sci. USA 101:4164-4169.
18. Bailey et al. (2016) Genomic analyses identify molecular subtypes of pancreatic cancer. Nature 531:47-52.
19. The Cancer Genome Atlas Research Network (2017) Integrated genomic characterization of pancreatic ductal adenocarcinoma. Cancer Cell 32:185-203.
20. Aung et al. (2018) Genomics-driven precision medicine for advanced pancreatic cancer: early results from the COMPASS trial. Clin. Cancer Res. 24:1344-1354.
21. Martens et al. (2019) Different shades of pancreatic ductal adenocarcinoma, different paths towards precision therapeutic applications. Ann. Oncol. 30:1428-1436.
22. Connor et al. (2019) Integration of genomic and transcriptional features in pancreatic cancer reveals increased cell cycle progression in metastases. Cancer Cell 35:267-282.
23. Subramanian et al. (2005) Gene set enrichment analysis: a knowledge-based approach for interpreting genome-wide expression profiles. Proc. Natl Acad. Sci. USA 102:15545-15550.
24. Liu et al. (2018) An integrated TCGA pan-cancer clinical data resource to drive high-quality survival outcome analytics. Cell 173:400-416.
25. Puleo et al. (2018) Stratification of pancreatic ductal adenocarcinomas based on tumor and microenvironment features. Gastroenterology 155:1999-2013.
26. Collisson et al. (2019) Molecular subtypes of pancreatic cancer. Nat. Rev. Gastroenterol. Hepatol. 16:207-220.
27. Corces et al. (2018) The chromatin accessibility landscape of primary human cancers. Science 362:eaav1898.
28. Hoadley et al. (2018) cell-of-origin patterns dominate the molecular classification of 10,000 tumors from 33 types of cancer. Cell 173:291-304.
29. The Cancer Genome Atlas Network (2012) Comprehensive molecular portraits of human breast tumours. Nature 490:61-70.
30. Wei et al. (2012) Insights into pancreatic cancer etiology from pathway analysis of genome-wide association study data. PLoS ONE 7:e46887.
31. Weng et al. (2006) PSGR2, a novel G-protein coupled receptor, is overexpressed in human prostate cancer. Int. J. Cancer 118:1471-1480.
32. Wang et al. (2006) The prostate-specific G-protein coupled receptors PSGR and PSGR2 are prostate cancer biomarkers that are complementary to alphamethylacyl-CoA racemase. Prostate 66:847-857.
33. Weber et al. (2018a) Characterization of the olfactory receptor OR10H1 in human urinary bladder cancer. Front. Physiol. 9:456.
34. Weber et al. (2018b) Olfactory receptors as biomarkers in human breast carcinoma tissues. Front. Oncol. 8:33.
35. Sur & Taipale (2016) The role of enhancers in cancer. Nat. Rev. Cancer 16:483-493.
36. Zhong Y, Wan Y W, Pang K, Chow L M, Liu Z. Digital sorting of complex tissues for cell type-specific gene expression profiles. BMC bioinformatics. 2013;14:89. doi: 10.1186/1471-2105-14-

[symbol]

It will be understood that various details of the presently disclosed subject matter can be changed without departing from the scope of the presently disclosed subject matter. Furthermore, the foregoing description is for the purpose of illustration only, and not for the purpose of limitation.

1. A method for de novo deconvoluting a dataset with multiple samples and/or estimating compartment weight for a single sample, the method comprising applying the following steps to a dataset with multiple samples or to a single sample:
using a nonnegative matrix factorization (NMF) algorithm to calculate a NMF seed training configured to calculate a stable gene weight seed (W″), wherein W″ is a 50*×\tilde{K} matrix of 50*\tilde{K} rows of genes and \tilde{K} columns of factor, completed for R repetitions resulting in R data partitions;
executing an NMF algorithm for each of R data partitions;
calculating a gene weight matrix for a current number (K) of factors; and
executing a final NMF algorithm to generate a gene weight matrix W′ (a 5000*\tilde{K} matrix of 5000 rows of genes and K columns of factors), and compartment weight matrix H′ (a \tilde{K}×M matrix of \tilde{K} rows of factors and M columns of samples),
whereby the dataset is deconvoluted and/or a compartment weight is estimated.

2. The method of claim 1, wherein R repetitions is about 10,000 or greater.

3. The method of claim 1, wherein the NMF algorithm for each of the R data partitions comprises an unsupervised NMF executed with at least 20 randomly initialized instances of NMF using a multiplicative update NMF solver for ten steps using a built-in NMF function in MATLAB (R2017b).

4. The method of claim 1, further comprising calculating a consensus matrix, wherein the consensus matrix represents a frequency of the genes to be determined as the top genes, and wherein the consensus matrix is used for hierarchical clustering to yield \tilde{K} gene clusters.

5. The method of claim 1, wherein calculation of the gene weight matrix comprises ranking genes in a matrix in descending order of a weight difference between a current factor weight and a largest weight in the rest of the factors, wherein a top 50 genes for any factor are recorded in a gene-by-gene consensus matrix C (50*\tilde{K}×50*\tilde{K}).

6. The method of claim 1, further comprising using a non-negative least square (NNLS) algorithm to find:
arg minₕ_{ᵢ}∥W′\tilde{h}ᵢ−a′ᵢ∥₂subject to \tilde{h}ᵢ≥0,
where \tilde{h}ᵢ is the ith column/sample in \tilde{H} (a \tilde{K}×M matrix) to be determined, a′ᵢ is the ith column/sample in A′ (a 5000×M matrix, wherein \tilde{H} is considered to record the final compartment weights for each factor at the current number of factors (\tilde{K}) from the de novo deconvolution.

7. The method of claim 6, further comprising using a NNLS algorithm to find:
arg min_{w}_{ⱼ}∥\tilde{H}\tilde{w}ⱼ−aⱼ∥₂subject to \tilde{W}ⱼ24 0,
where \tilde{w}ⱼ is the jth row/gene in \tilde{W} (a N×\tilde{K} matrix) to be determined, and aⱼ is the jth row/gene in A (a N×M matrix), wherein \tilde{W} is considered to record the final gene weights for each factor at the current number of factors (\tilde{K}) from the de novo deconvolution, wherein gene weight matrix (\tilde{W}) is further used for ranking of genes, calculation of factor scores, and/or annotation of compartments.

8. The method of claim 1, wherein the dataset comprises RNA sequence and/or gene expression data, optionally tumor RNA sequence and/or gene expression data, optionally ATACseq data.

9. The method of claim 8, wherein the tumor is any tumor, optionally where the tumor is a pancreatic tumor, a prostate cancer, a bladder cancer, or a breast cancer.

10. The method of claim 9, wherein the tumor is from a subject, optionally wherein the subject is a human subject.

11. A method of diagnosing a cancer and/or identifying a tumor type in a subject where the tumor cannot be identified through pathology, the method comprising:
providing a sample from a subject to be diagnosed; and
performing the method of claim 1 on the sample from the subject,
wherein a cancer in the subject is diagnosed, and/or a tumor type in the subject is identified.

12. The method of claim 11, wherein the subject has pancreatic ductal adenocarcinoma (PDAC).

13. The method of claim 11, wherein the subject is a human.

14. The method of claim 11, wherein the diagnosing of a cancer and/or identifying a tumor type further comprises calculating compartment weights to perform binary classification of tumor subtypes.

15. The method of claim 11, further comprising identifying one or more compartments within a cancer or tumor in the subject.

16. The method of claim 11, further comprising predicting a prognosis of an identified tumor type, and/or predicting efficacy of a treatment for an identified tumor type.

17. A method of estimating and/or identifying a cellular compartment in a tumor or cancer, the method comprising:
providing a tumor or cancer tissue sample; and
performing the method of claim 1 on the tissue sample,
wherein a cellular compartment in a tumor or cancer is identified.

18. The method of claim 17, wherein the tumor or cancer tissue sample is from a subject, optionally from a human subject.

19. The method of claim 17, wherein the tumor or cancer tissue sample is from any solid or hematologic malignancy and/or any liquid biopsy.

20. The method of claim 17, wherein the tumor or cancer tissue sample comprises a pancreatic cancer.
